SEQUENCE LISTING (38 sequences)

SEQ ID NO: 1 - DNA, 18 bp, Artificial Sequence
  Oligonucleotide Primer for F3 2019-nCoV N-gene
aacacaagctttcggcag

SEQ ID NO: 2 - DNA, 22 bp, Artificial Sequence
  Oligonucleotide Primer for B3 2019-nCoV N-gene
gaaatttggatctttgtcatcc

SEQ ID NO: 3 - DNA, 41 bp, Artificial Sequence
  Oligonucleotide Primer for BIP 2019-nCoV N-gene
tgcggccaatgtttgtaatcagccaaggaaattttggggac

SEQ ID NO: 4 - DNA, 39 bp, Artificial Sequence
  Oligonucleotide Primer for FIP 2019-nCoV N-gene
cgcattggcatggaagtcactttgatggcacctgtgtag

SEQ ID NO: 5 - DNA, 19 bp, Artificial Sequence
  Oligonucleotide Primer for LF 2019-nCoV N-gene
ttccttgtctgattagttc

SEQ ID NO: 6 - DNA, 18 bp, Artificial Sequence
  Oligonucleotide Primer for LB 2019-nCoV N-gene
accttcgggaacgtggtt

SEQ ID NO: 7 - DNA, 18 bp, Artificial Sequence
  Oligonucleotide Primer for F3 2019-nCoV E-gene
ccgacgacgactactagc

SEQ ID NO: 8 - DNA, 23 bp, Artificial Sequence
  Oligonucleotide Primer for B3 2019-nCoV E-gene
agagtaaacgtaaaaagaaggtt

SEQ ID NO: 9 - DNA, 41 bp, Artificial Sequence
  Oligonucleotide Primer for BIP 2019-nCoV E-gene
acctgtctcttccgaaacgaatttgtaagcacaagctgatg

SEQ ID NO: 10 - DNA, 41 bp, Artificial Sequence
  Oligonucleotide Primer for FIP 2019-nCoV E-gene
ctagccatccttactgcgctactcacgttaacaatattgca

SEQ ID NO: 11 - DNA, 19 bp, Artificial Sequence
  Oligonucleotide Primer for LF 2019-nCoV E-gene
tcgattgtgtgcgtactgc

SEQ ID NO: 12 - DNA, 19 bp, Artificial Sequence
  Oligonucleotide Primer for LB 2019-nCoV E-gene
tgagtacataagttcgtac

SEQ ID NO: 13 - DNA, 16 bp, Artificial Sequence
  SARS-CoV-2 N gDNA-1 flanked by /5Phos/ and /3Phos/
atgcgcgacattccga

SEQ ID NO: 14 - DNA, 16 bp, Artificial Sequence
  SARS-CoV-2 N gDNA-2 flanked by /5Phos/ and /3Phos/
agaacgctgaagcgct

SEQ ID NO: 15 - DNA, 16 bp, Artificial Sequence
  SARS-CoV-2 N gDNA-3 flanked by /5Phos/ and /3Phos/
ccccagcgcttcagcg

SEQ ID NO: 16 - DNA, 16 bp, Artificial Sequence
  SARS-CoV-2 E gDNA-1 flanked by /5Phos/ and /3Phos/
tggctagtgtaactag

SEQ ID NO: 17 - DNA, 16 bp, Artificial Sequence
  SARS-CoV-2 E gDNA-2 flanked by /5Phos/ and /3Phos/
tggctagtgtaactag

SEQ ID NO: 18 - DNA, 16 bp, Artificial Sequence
  SARS-CoV-2 E gDNA-3 flanked by /5Phos/ and /3Phos/
ttgctttcgtggtatt

SEQ ID NO: 19 - DNA, 17 bp, Artificial Sequence
  SARS-CoV-2 N reporter probe flanked by /56-FAM/ on the 5′ end and /3BHQ_1/ on the 3′ end
ttccgaagaacgctgaa

SEQ ID NO: 20 - DNA, 17 bp, Artificial Sequence
  SARS-CoV-2 E reporter probe flanked by /56-ROXN/ on the 5′ end and /3BHQ_2/ on the 3′ end
aactagcaagaatacca

SEQ ID NO: 21 - DNA, 25 bp, Artificial Sequence
  Oligonucleotide Primer for N-gene-FWD IVT
aattctaatacgactcactataggg

SEQ ID NO: 22 - DNA, 21 bp, Artificial Sequence
  Oligonucleotide Primer for N-gene-REV IVT
cacagtttgctgtttcttctg

SEQ ID NO: 23 - DNA, 25 bp, Artificial Sequence
  Oligonucleotide Primer for E-gene-FWD IVT
aattctaatacgactcactataggg

SEQ ID NO: 24 - DNA, 22 bp, Artificial Sequence
  Oligonucleotide Primer for E-gene-REV IVT
ctgccatggctaaaattaaagt

SEQ ID NO: 25 - DNA, 500 bp, Artificial Sequence
  dsDNA Gene Fragments Sequence (5′ to 3′) for SARS-CoV-2 N-gene
aattctaatacgactcactataggggtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtg

SEQ ID NO: 26 - DNA, 500 bp, Artificial Sequence
  dsDNA Gene Fragments Sequence (5′ to 3′) for SARS-CoV-2 E-gene
aattctaatacgactcactatagggagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcag

SEQ ID NO: 27 - DNA, 16 bp, Artificial Sequence
  SARS-CoV-2 N gDNA-32 flaked by /5phos/
tcgcgaagtcgcaaga

SEQ ID NO: 28 - DNA, 16 bp, Artificial Sequence
  SARS-CoV-2 N gDNA-1 flaked by /5phos/
agccttacagcgcgta

SEQ ID NO: 29 - DNA, 55 bp, Artificial Sequence
  SARS-CoV-2 N 5′ to 3′
aatttgcccccagcgcttcagcgttcttcggaatgtcgcgcattggcatggaagt

SEQ ID NO: 30 - DNA, 55 bp, Artificial Sequence
  SARS-CoV-2 N 3′ to 5′
ttaaacgggggtcgcgaagtcgcaagaagccttacagcgcgtaaccgtaccttca

SEQ ID NO: 31 - DNA, 16 bp, Artificial Sequence
  SARS-CoV-2 N Secondary gDNA
tcagcgttcttcggaa

SEQ ID NO: 32 - DNA, 17 bp, Artificial Sequence
  SARS-CoV-2 N Fluorescent reporter flanked by BHQ1 and FAM
aagtcgcaagaagcctt

SEQ ID NO: 33 - DNA, 16 bp, Artificial Sequence
  SARS-CoV-2 E gDNA-2 flaked by /5phos/
aaagcaccataagaac

SEQ ID NO: 34 - DNA, 16 bp, Artificial Sequence
  SARS-CoV-2 E gDNA-1 flaked by /5phos/
gatcaatgtgatcggt

SEQ ID NO: 35 - DNA, 55 bp, Artificial Sequence
  SARS-CoV-2 E 5′ to 3′
ctttttcttgctttcgtggtattcttgctagttacactagccatccttactgcgc

SEQ ID NO: 36 - DNA, 55 bp, Artificial Sequence
  SARS-CoV-2 E 3′ to 5′
gaaaaagaacgaaagcaccataagaacgatcaatgtgatcggtaggaatgacgcg

SEQ ID NO: 37 - DNA, 16 bp, Artificial Sequence
  SARS-CoV-2 E Secondary gDNA
ggtattcttgctagtt

SEQ ID NO: 38 - DNA, 17 bp, Artificial Sequence
  SARS-CoV-2 E Fluorescent reporter flanked by BHQ2 and ROX
accataagaacgatcaa

(19) United States
(12) Patent Application Publication
(10) Pub. No.: US 2024/0011082 A1
(43) Pub. Date: Jan. 11, 2024

(54) RAPID, ACCURATE, SCALABLE AND PORTABLE TESTING (SPOT) SYSTEM

(71) Applicant: THE BOARD OF TRUSTEES OF THE UNIVERSITY OF ILLINOIS, Urbana, IL (US)

(72) Inventors: Huimin ZHAO, Champaign, IL (US); Guanhua XUN, Urbana, IL (US); Stephan LANE, Urbana, IL (US)

(73) Assignee: THE BOARD OF TRUSTEES OF THE UNIVERSITY OF ILLINOIS, Urbana, IL (US)

(21) Appl. No.: 18/023,107

(22) PCT Filed: Sep. 8, 2021
(86) PCT No.: PCT/US21/49481

§ 371 Date: Feb. 24, 2023

Related U.S. Application Data
(60) Provisional application No. 63/075,744, filed on Sep. 8, 2020.
(60) Provisional application No. 63/155,127, filed on Mar. 1, 2021.

(51) Int. Cl. C12Q 1/6844; C07K 14/195; C12Q 1/70

(52) U.S. Cl. CPC C12Q 1/6846; C07K 14/195; C12Q 1/701; G01N 2800/26

(57) ABSTRACT

The present disclosure provides assay containers, sets of containers, portable diagnostic devices, methods of detecting target nucleic acid molecules in a sample, and methods of diagnosing infections, such as COVID-19 infections, in a subject.

### PRIORITY

This application claims the benefit of U.S. Ser. No. 63/075,744, filed on Sep. 8, 2020, and U.S. Ser. No. 63/155,127, filed on Mar. 1, 2021, which are incorporated by reference herein in their entirety.

### BACKGROUND

The implementation of LAMP reactions to specifically and accurately detect the presence of target nucleic acid molecules in a sample is limited by the occurrence of non-specific amplification, which significantly increases the rate of false positives and precludes their use as a diagnostic assay. Therefore, there is a need for the development of improved methods that overcome those limitations, that can be used for detecting nucleic acids in samples.

### SUMMARY

Provided herein are assay containers, sets of containers, portable diagnostic devices, methods of detecting target nucleic acid molecules in a sample, and methods of diagnosing an infection, such as a COVID-19 infection, in a subject.

An embodiment provides an assay container comprising: a removable barrier separating an upper chamber from a lower chamber, the upper chamber comprising one or more sets of primers for the amplification of one or more target nucleic acid molecules and a reverse transcriptase for a reverse transcriptase-loop-mediated isothermal amplification (RT-LAMP) reaction, and the lower chamber comprising a hyperthermophilic archaeon Argonaute protein, one or more sets of single-stranded guide DNA (gDNA) specific for one or more target nucleic acid molecules, and one or more reporter molecules for a hyperthermophilic archaeon Argonaute protein-based target sequence detection reaction.

The one or more sets of primers can comprise two or more oligonucleotides. The two or more oligonucleotides can comprise two or more of SEQ ID NOs:1-6 or SEQ ID NOs:7-12. The hyperthermophilic archaeon Argonaute protein can be an Argonaute protein from, for example, Pyrococcus furiosus, Thermococcus thioreducens, Thermococcus onnurineus, Thermococcus eurythermalis, Methanocaldococcus bathoardescens, Methanocaldococcus sp. FS406-22, Methanocaldococcus fervens, Methanocaldococcus jannaschii, Methanotorris formiscicus, Ferroglobus placidus, Archaeoglobus fulgidus, Sulfolobus sp. (e.g., S. solfataricus), Methanopyrus kandleri, or Thermogladius cellulolyticus. The hyperthermophilic archaeon Argonaute protein can be a Pyrococcus furiosus Argonaute protein (PfAgo). The one or more sets of gDNA can comprise two or more gDNAs. The two or more gDNAs can comprise two or more of SEQ ID NOs:13-15 or SEQ ID NOs:16-18. The reporter molecule can comprise a nucleic acid molecule having complementarity to the one or more target nucleic acid molecules and a fluorescent moiety. A nucleic acid molecule having complementarity to the target nucleic acid molecule can comprise SEQ ID NO:19 or SEQ ID NO:20. The fluorescent moiety can be carboxyfluorescein (FAM) or carboxyrhodamine (ROX). The one or more target nucleic acid molecules can be one or more SARS-CoV-2 nucleic acid molecules. The SARS-CoV-2 nucleic acid molecule can be an envelope (E) nucleic acid molecule, a nucleoprotein (N) nucleic acid molecule, or a combination of both. The container can be a capillary tube. The removable barrier can be a meltable plug.

Another embodiment provides a set of containers comprising a sample preparation container, and the assay container described herein.

The sample preparation container can comprise a DNA extraction solution. The set of containers can further comprise a transfer container.

An embodiment provides a portable diagnostic device comprising a) a heating module, b) a fluorometry module, c) the assay container described herein within an opening to hold the assay container, and d) an LCD screen.

The heating module can comprise a copper heat block wrapped with nichrome wire and an integrated fan. The copper heat block can be configured to allow temperature control, and the integrated fan can be configured to enable cooling. The fluorometry module can comprise an LED board, an excitation bandpass filter, a photodiode board, emission bandpass filters, and a motherboard comprising a microcontroller. The emission bandpass filters can be mounted perpendicular to the capillary and filter light to the photodiodes. The LED board can comprise a blue LED located beneath the excitation bandpass filter, the blue LED can shine light into a hole at the bottom of the copper heat block and into the assay container. The device can further comprise a transimpedance amplifier configured to convert photodiode current into voltage, which can be read by the microcontroller. The photodiode board can comprise one or more photodiodes secured behind the emission bandpass filters. The device can further comprise a rechargeable and removable lithium-ion battery. The device can further comprise a cap covering the device to block excess light.

Another embodiment provides a method of detecting one or more target nucleic acid molecules in a sample comprising a) amplifying one or more target nucleic acid molecules using a RT-LAMP reaction to produce amplified target nucleic acid molecules, b) contacting the amplified target nucleic acid molecules with a hyperthermophilic archaeon Argonaute protein, one or more sets of gDNA specific for the one or more target nucleic acid molecules, and one or more reporter molecules, and c) detecting fluorescence in the sample.

Steps a), b) and c) can be performed in a single assay container. Steps a), b) and c) can be performed in the assay container described herein. Target nucleic acid molecules can be one or more SARS-CoV-2 nucleic acid molecules. The one or more SARS-CoV-2 nucleic acid molecules can be envelope (E) nucleic acid molecules, nucleoprotein (N) nucleic acid molecules, or a combination of both. Step a) can comprise a) contacting the sample with the one or more sets of primers specific for amplification of the target nucleic acid molecules and a reverse transcriptase, and b) heating the sample at 60° C. to 65° C. for 5 or more minutes. The one or more sets of primers can comprise six oligonucleotides selected from SEQ ID NOs:1-6 and SEQ ID NOs:7-12. Step b) can comprise heating the sample at 90° C. to 100° C. The one or more sets of gDNA can comprise three gDNAs selected from SEQ ID NOs:13-15 and SEQ ID NOs:16-18. The one or more reporter molecules can comprise a nucleic acid molecule having complementarity to the target nucleic acid molecules and a fluorescent moiety. The nucleic acid molecules having complementarity to the target nucleic acid molecule can comprise SEQ ID NO:19 or SEQ ID NO:20. The fluorescent moiety can be FAM or ROX.

An additional embodiment provides a method of diagnosing a COVID-19 infection in a subject comprising detecting one or more SARS-CoV-2 nucleic acid molecules in a sample from the subject comprising: a) loading the sample into an upper chamber of a assay container comprising a removable barrier separating an upper chamber from a lower chamber, the upper chamber comprising one or more set of primers specific for the amplification of one or more SARS-CoV-2 nucleic acid molecules and the reverse transcriptase for a RT-LAMP reaction and the lower chamber comprising a hyperthermophilic archaeon Argonaute protein, one or more sets of gDNA specific for one or more SARS-CoV-2 nucleic acid molecules, and one or more reporter molecules for a hyperthermophilic archaeon Argonaute protein-based target sequence detection reaction, b) inserting the assay container into a portable diagnostic device comprising: a heating module, a fluorometry module, an opening to insert and hold the assay container, and an LCD screen, c) heating the sample at 60° C. to 65° C. for 5 minutes or more; d) heating the sample at 90° C. to 100° C. for 3 minutes or more; and e) reading a diagnostic test result on the LCD screen.

The one or more sets of primers can comprise six oligonucleotides selected from SEQ ID NOs:1-6 and SEQ ID NOs:7-12. The one or more sets of gDNA can comprise three gDNAs selected from SEQ ID NOs:13-15 and SEQ ID NOs:16-18. The reporter molecule can comprise a nucleic acid molecule having complementarity to the SARS-CoV-2 nucleic acid molecules and a fluorescent moiety. The nucleic acid molecule having complementarity to the SARS-CoV-2 nucleic acid molecule can comprise SEQ ID NO:19 or SEQ ID NO:20. The fluorescent moiety can be FAM or ROX. The method can further comprise contacting the sample with a DNA extraction solution in a sample preparation container and heating the sample in the sample preparation container prior to a). The sample can be a saliva sample.

Provided herein are assay containers, sets of containers, portable diagnostic devices, methods of detecting target nucleic acid molecules in a sample, and methods of diagnosing a COVID-19 infection in a subject.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIG. 1A-1B illustrate an overview of the SPOT system. FIG. 1A shows an overall workflow using capillaries and the SPOT device. The dashed lines represent excitation spectrum of FAM and ROX, respectively, while emission spectrums are shown solid lines. Inset: Device and consumables needed to run the SPOT system, including a 3D printed portable detection device, a pre-made sample pretreatment capillary 1, a pre-made viral RNA detection capillary 2, and collection capillaries. A quarter coin is placed for scale (diameter: 24.26 mm). FIG. 1B shows genome map showing primers, gDNAs and SPOT mechanism. RT-LAMP primers are indicated by black rectangles. Enclosed “P” represents the phosphate group.

FIG. 2A-2D illustrates the mechanism of SPOT reaction and cleavage efficiency. FIG. 2A illustrates the gDNA-1 and gDNA-2 for N gene generate the secondary gDNA by cleaving the sense strand of N gene amplicon. The gDNA-3 helps to release the secondary gDNA from the primary cleavage. The secondary gDNA targets to the complementary fluorescent moiety to mediate the cleavage on reporter by PfAgo. FIG. 2B illustrates the gDNA-1 and gDNA-2 for E gene generate the secondary gDNA by cleaving the sense strand of E gene amplicon. The gDNA-3 helps to release the secondary gDNA from the primary cleavage. The secondary gDNA targets to the complementary fluorescent moiety to mediate the cleavage on reporter by PfAgo. For the N gene, the gDNA-2 is SEQ ID NO:27, the gDNA1 is SEQ ID NO:28, the N gene 5′ to 3′ sequence is SEQ ID NO:29, the N gene 3′ to 5′ sequence is SEQ ID NO:30, the gDNA-3 is SEQ ID NO:15, the secondary gDNA is SEQ ID NO:31, the fluorescent reporter is SEQ ID NO:32. For the E gene, the gDNA-2 is SEQ ID NO:33, the gDNA1 is SEQ ID NO:34, the E gene 5′ to 3′ sequence is SEQ ID NO:35, the N gene 3′ to 5′ sequence is SEQ ID NO:36, the gDNA-3 is SEQ ID NO:18, the secondary gDNA is SEQ ID NO:37, the fluorescent reporter is SEQ ID NO:38. FIG. 2C illustrates the comparison of cleavage efficiency between combinations of gDNAs (n=3). FIG. 2D illustrates the optimization of the fluorescence-based nucleic acid detection reaction. Three candidate enzymes were added at the same amount. TtAgo mediated reaction was incubated at 80° C. without manganese ion (Mn²⁺). PfAgo mediated reaction was performed at 95° C. with manganese ion (Mn²⁺). Optimized PfAgo mutant, which shows cleavage activity in the presence of magnesium ion (Mg²⁺), was added to the reaction at 95° C. (n=3).

FIG. 3A-3F illustrates the optimization and validation of the SPOT assay. FIG. 3A illustrates the LOD using RNA samples. Reactions were performed in a thermocycler and fluorescence values were measured on a qPCR machine (n=3) using SARS-CoV-2 E gene IVT RNA. FIG. 3B illustrates the LOD using virus-spiked saliva samples with only N gene corresponding primers. Reactions were performed in a thermocycler and fluorescence values were measured using a qPCR machine (n=3). FIG. 3C illustrates the LOD using virus-spiked saliva samples with only E gene corresponding primers. Reactions were performed in a thermocycler and fluorescence values were measured using a qPCR machine (n=3). FIG. 3D illustrates the multiplexity of the assay. Both N gene and E gene corresponding primers were added. Reactions were performed in a thermocycler and fluorescence values were measured using a qPCR machine (n=3). FIG. 3E illustrates the PfAgo detection components affect LAMP reaction. Lanes from left to right: LAMP positive control, 0.5 mM manganese ion, 1 mM gDNAs, 0.3 μM fluorophore-quencher labeled reporter probe added, 5.5 μM PfAgo. FIG. 3F illustrates the strategy for compartmenting two reactions: the upper chamber (liquid represents RT-LAMP reaction) and the lower chamber (liquid represents mixture of PfAgo and manganese ion) are separated by paraffin wax (melting point above 70° C.).

FIG. 4 illustrates the optimization of saliva sample pre-treatment time. Saliva samples spiked with 0, 1.75, 8.75, 17.5, and 43.8 copies of gamma-irradiated SARS-CoV-2 virus were mixed with QuickExtract™ DNA Extraction Solution at a 1:1 ratio and heated at 95° C. for 1 min, 5 min, 10 min and 15 min, respectively (n=3).

FIG. 5A-5B illustrates additives for enhancing viral genome release. Tween™ 20 (polysorbate) and DNA extraction solution were explored for their ability to enhance SARS-CoV-2 detection. FIG. 5A illustrates when 0.5% Tween20™ (polysorbate) was added to a saliva sample spiked with gamma irradiated SARS-CoV-2 virus and heated at 95° C. for 5 min (n=3). FIG. 5B illustrates when 50% DNA extraction solution (Lucigen, Inc.) was added to a saliva sample spiked with gamma irradiated SARS-CoV-2 virus and heated at 95° C. for 5 min (n=3).

FIG. 6 illustrates the optimization of the LAMP reaction time. Saliva samples spiked with 43.8 copies of gamma-irradiated SARS-CoV-2 virus were added into the RT-LAMP reaction and incubated at 63° C. for different lengths of time (n=3).

FIG. 7 illustrates the optimization of PfAgo cleavage reaction time. A saliva sample spiked with 43.8 copies of gamma-irradiated SARS-CoV-2 virus was added into the RT-LAMP reaction mixture and incubated at 63° C. for 30 min. PfAgo was then added into the RT-LAMP reaction product and the fluorescence signal was determined by a real-time PCR machine with the following program: 20 sec. at 95° C., 5 sec. at 25° C. for capturing the fluorescent signal, a total of 30 cycles (n=3).

FIG. 8A-D illustrates the design and validation of the SPOT device. FIG. 8A illustrates the rendering of the fully enclosed SPOT device along with a partially exploded view. Users press buttons to power on the device (1) and start detection reactions (2) and read results on the integrated LCD screen (3). Reaction progress is displayed on the LCD screen and indicator LEDs (4). The SPOT device can export data via a USB cable (5) and be recharged. An electronics cap covers the optical and thermal modules, which are comprised of a capillary in a copper heat block, a cooling fan, a photodiode PCB placed securely behind emission bandpass filters, a structural platform, and an excitation bandpass filter covering a 470 nm excitation LED and PCB. The reaction timings, temperature, and results interpretation are performed on a main circuit board powered by a rechargeable battery, contained within a main enclosure capped with front and back covers. FIG. 8B illustrates the capability of performing detection by using the SPOT device. SPOT assays using gamma-irradiated virus spiked samples were run on both the SPOT device (open dots for N gene) and a thermocycler (black dots), then fluorescence values were measured on the SPOT device. SPOT assays performed on the SPOT device using gamma-irradiated virus spiked samples at 0, 43.8, 175, and 1750 viral copies per reaction (n=3). FIG. 8C illustrates the capability of performing detection by using the SPOT device. SPOT assays using gamma-irradiated virus spiked samples were run on both the SPOT device (slash dots for E gene) and a thermocycler (black dots), then fluorescence values were measured on the SPOT device. SPOT assays performed on the SPOT device using gamma-irradiated virus spiked samples at 0, 43.8, 175, and 1750 viral copies per reaction (n=3). FIG. 8D illustrates the validation using clinical saliva samples in a blinded test. Patient sample fluorescence data from SPOT device readout. Clinical samples from 104 patients were collected from the campus of University of Illinois at Urbana-Champaign. Signal intensities from SPOT were measured using a 2× amplifier gain on photodiode signal, with a positive threshold set at 5×s.d. above background. “PC” indicates the positive control sample, “NC” indicates the negative control sample.

FIG. 9 illustrates the temperature profile of the thermal module in the SPOT device. The SPOT device heated the capillary to 63° C. and 95° C. in 25 seconds and 30 seconds, respectively. (Temperature data was collected from a temperature sensor placed inside a capillary).

FIG. 10A-B illustrates the comparison of sensitivity of fluorescence quantification between SPOT device and qPCR machine. To analyze SPOT assay results using SPOT device fluorescence quantification, the SPOT assays were performed on a thermocycler and then transferred to a capillary for fluorescent quantification using the SPOT device. FIG. 10A illustrates in the N gene associated FAM fluorescence. FIG. 10B illustrates the E gene associated ROX fluorescence. As a comparison, those samples were also measured on a qPCR machine (QuantStudio 3 RT-PCR system), marked in slash dots in both FIG. 10A and FIG. 10B. SPOT assays were performed using gamma irradiated virus spiked saliva with initial copies of viral particles at 0, 43.8, 175, and 1750 viral copies per reaction (n=3).

FIG. 11 illustrates the stability of prefabricated reaction capillary. Reaction capillaries were prepared using LAMP and PfAgo chambers and stored at −20° C. The capillaries were taken out to measure the activity over time (n=3).

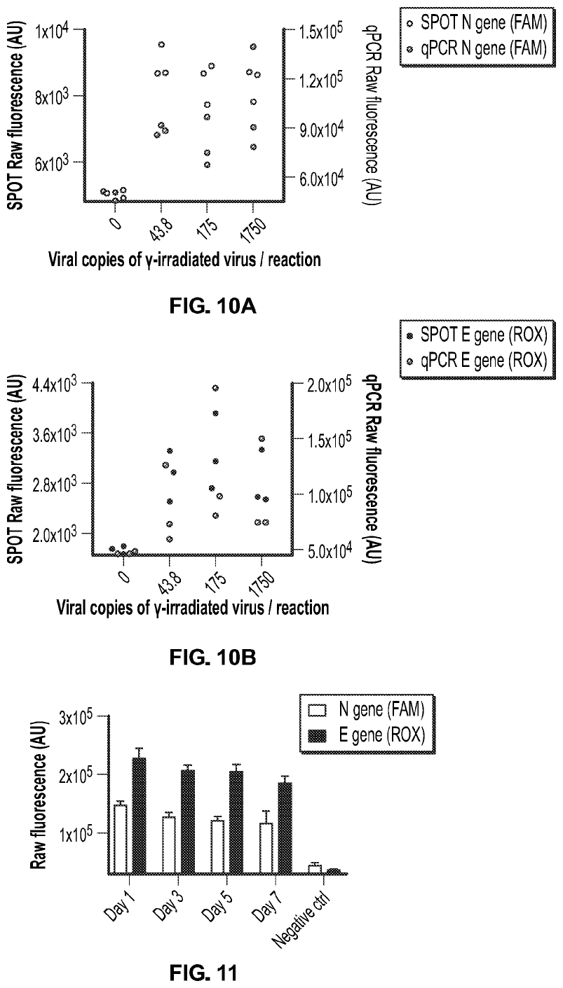

FIG. 12 illustrates the perspective of the SPOT device. By connecting the SPOT system with networked devices, such as laptops, tablets, and smartphones, the advanced capabilities such as connectivity, databasing, and data sharing enable better global pandemic controlling, platform of telemedicine, and decentralized testing.

### DETAILED DESCRIPTION

Many modifications and other embodiments of assay containers, devices and methods of use thereof described herein will come to mind to one of skill in the art having the benefit of the teachings presented in the foregoing descriptions and the associated drawings. Therefore, it is to be understood that the methods and compositions are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art.

Overview

Rapid, accurate, and scalable testing systems for the detection of nucleic acids, including the detection of nucleic acids of pathogens such as SARS-CoV-2 are needed in the art. For example, COVID-19 diagnosis is essential to control SARS-CoV-2, the novel coronavirus causing COVID-19 pandemic. The diagnosis of COVID-19 mainly relies on the detection of the coronavirus RNA. The gold standard testing system approved by US Centers for Disease Control and Prevention (CDC) comprises a thermocycler and a bioassay based on the quantitative reverse transcriptase-polymerase chain reaction (qRT-PCR). Other existing testing systems are based on reverse transcriptase-loop-mediated isothermal amplification (RT-LAMP). Although RT-LAMP-based testing systems have eliminated the requirement for expensive and bulky thermocyclers, they have only limited portability due to their requirements for heat blocks or water baths. Moreover, as test results are read from a lateral flow strip, these systems produce a qualitative rather than quantitative result.

Both qRT-PCR and RT-LAMP based assays have their advantages and disadvantages. The former is widely used for virus identification with high sensitivity and specificity, but its analysis requires various equipment and educated analysts, which is only possible in a laboratory. It normally takes 4-6 hours for testing and over 24 hours to return the result to the patient. The latter is a highly sensitive nucleic acid amplification method that can often detect small numbers of DNA or RNA templates within half an hour. However, the requirement for high temperature limits its compatibility with other enzymes, forcing CRISPR-Cas based methods to run amplification and detection steps separately or optimize the Cas protein to survive at high temperatures. Although some Cas proteins can function at 60° C., this temperature is suboptimal for both the Cas protein and Bst 2.0, the DNA polymerase used in LAMP, dramatically decreasing their activity and prolonging the detection period. Separating the amplification and detection steps into a two-step reaction increases the risk of contamination due to the extremely high sensitivity of RT-LAMP. Moreover, existing RT-LAMP-based methods can only detect one gene at a time, reducing the accuracy of diagnosis. Most importantly, because all current COVID-19 tests require many complicated operation steps, scaling up requires trained personnel and specialized tools.

Provided herein is a Scalable and Portable Testing (SPOT) system comprising a rapid, highly sensitive, and accurate assay and a battery-powered portable device for the detection of nucleic acids, including RNA viruses such as SARS-CoV-2. A SPOT system combines RT-LAMP with an Argonaute protein (Ago) from a hyperthermophilic archaeon such as Pyrococcus furiosus (PfAgo) capable of precise recognition and cleavage of a target DNA at high temperature, such as 95° C. as directed by small single strand DNA (ssDNA) as guide DNA (gDNA). Due to the multi-turnover activity of Ago protein such as PfAgo, its secondary cleavage mechanism can be harnessed for specific, sensitive, and multiplex nucleic acid detection. For COVID-19 samples, although nasopharyngeal swab and nasal swab samples have been recommended for detection of SARS-CoV-2, saliva samples are a more attractive alternative due to the ease, safety and non-invasive nature of its collection, and its relatively high viral load during the first week of infection. These benefits enable a saliva sample to be an ideal specimen for reliable and rapid self-detection without professional supervision. While current CRISPR-based detection systems normally require 50 min for testing, Ago, such as PfAgo, can dramatically speed up the detection process by requiring only 3-5 minutes for cleavage of amplified products, thereby shortening the total time for testing to less than 30 min. Moreover, successful Ago, such as PfAgo, detection requires at least two sequence-specific cleavages, endowing the SPOT system with high specificity and the ability for multiplexing.

The need for rapid, accurate, and scalable testing systems for the diagnosis of pathogen-induced diseases, such as COVID-19 diagnosis is clear and urgent. The Scalable and Portable Testing (SPOT) system described herein comprises a rapid, highly sensitive, and accurate assay and a battery-powered portable device for the detection of pathogen nucleic acids such as RNA viruses like SARS-CoV-2. The SPOT assay system can detect as low as 7.5 copies/reaction (0.19 copies/μL) of in vitro transcribed SARS-CoV-2 RNA and the N gene and E gene in a multiplexed reaction with the limit of detection (LOD) of 17.5 copies/reaction (0.44 copies/μL) and 43.8 copies/reaction (1.09 copies/μL), respectively, in SARS-CoV-2 virus-spiked saliva samples within 30 min. Moreover, the SPOT system was used to analyze 104 clinical saliva samples and identified 28/30 (93.3% accuracy) SARS-CoV-2 positive samples (100% accuracy if LOD is considered) and 73/74 (98.6% accuracy) SARS-CoV-2 negative samples. This combination of speed, accuracy, sensitivity, and portability will enable high-volume low-cost access to areas in need of urgent COVID-19 testing capabilities.

Assay Container

Provided herein are assay containers. An assay container can comprise a removable barrier separating a first chamber from a second chamber. The two chambers can be e.g., next to each other such that a first chamber can be a right chamber or a left chamber, and a second chamber can be a left chamber of a right chamber. Alternatively, the two chambers can be e.g., on top of each other, such that a first chamber can be an upper chamber or a lower chamber, and a second chamber can be a lower chamber or a lower chamber. Throughout the specification below, the two chambers are referred to as lower chamber and upper chamber, but the description below can also be applied to a left chamber and a right chamber.

An assay container can comprise a removable barrier separating an upper chamber from a lower chamber. An upper chamber can comprise one or more sets of primers for the amplification of one or more target nucleic acid molecules and a reverse transcriptase for a reverse transcriptase-loop-mediated isothermal amplification (RT-LAMP) reaction. A lower chamber can comprise a hyperthermophilic archaeon Argonaute protein, one or more sets of single-stranded guide DNA (gDNA) specific for one or more target nucleic acid molecules, and one or more reporter molecules for a hyperthermophilic archaeon Argonaute protein-based target sequence detection reaction.

An assay container can be any container that can be separated into an upper chamber and a lower chamber by a removable barrier. An assay container can include a generally tubular body, or any other suitable shape, with an open end. An assay container can include an aperture running lengthwise of the container that can accommodate the elements necessary to perform nucleic acid molecules amplification reactions, the elements necessary to perform hyperthermophilic archaeon Argonaute protein-based target sequence detection reactions, and a removable barrier. For example, the assay container can be a tube, such as a capillary tube, a well, or a chip.

An assay container can be made out of any material that is compatible with the conditions (e.g., temperature conditions) required to perform the nucleic acid molecule amplification and the hyperthermophilic archaeon Argonaute protein-based target sequence detection reactions. For example, the assay container can be made out of polypropylene, metal, glass, or any other suitable solid, nonabsorbent material, and can be partially or fully translucent for visibility. For example, the container can be a capillary tube.

An assay container can be separated into an upper chamber and a lower chamber by a removable barrier. The removable barrier can be any type of solid barrier that can be broken or otherwise removed from between the two chambers under certain conditions.

A non-limiting example of removable barrier is a meltable plug. A meltable plug can be made out of any material that can be in a solid state at certain ranges of temperatures, and that can be in a liquid (or melted state) at another range of temperature. For example, a meltable plug can be in a solid state below the temperature range required to perform nucleic acid molecule amplification reactions (e.g., any temperatures below 65° C., such as 0, 5, 10, 15, 20, 25, 30, 35, 40, 45, 50, 55 or 60° C.), and in a melted state at a temperature that is above the temperature range required to perform nucleic acid molecule amplification reactions, including at a temperature range required to perform hyperthermophilic archaeon Argonaute protein-based target sequence detection reactions (e.g., any temperature above 65° C., such as 70, 75, 80, 85, 90, 95, 100, 105° C. or more). That is the meltable plug can allow the nucleic acid amplification reaction to occur in the upper chamber without having the sample contacting the elements contained in the lower chamber. The barrier can be removed before performing the Argonaute protein-based target sequence detection reaction in the lower chamber such that the amplified material from the upper chamber moves to the lower chamber and contacts the elements contained in the lower chamber when the barrier is removed.

A meltable plug can be made of any wax such as animal waxes, vegetable waxes, mineral waxes or petroleum waxes. For example, a meltable plug can be made of beeswax, Chinese wax, lanolin (wool wax), shellac wax, spermaceti, bayberry wax, candelilla wax, carnauba wax, castor wax, esparto, Japan wax, jojoba oil, ouricury wax, rice bran wax, soy wax, tallow tree wax, ceresin waxes, montan wax, peat waxes, paraffin wax or microcrystalline wax. The meltable plug can be a paraffin wax plug.

Another example of a removable barrier is a solid physical barrier, such as a plastic, a silicone or a rubber plug. A plug can be made out of any similar or equivalent non-absorbent polymer that would provide a complete seal of an assay container (i.e., a complete separation between a lower chamber and an upper chamber), while remaining easily removable from the assay container. That is a removable plug can allow a nucleic acid amplification reaction to occur in an upper chamber without having the sample contacting the elements contained in a lower chamber. The barrier can be physically removed from an assay container before performing the Argonaute protein-based target sequence detection reaction in a lower chamber such that the amplified material from the upper chamber moves to the lower chamber and contacts the elements contained in the lower chamber when the barrier is removed. For example, a removable plug can be taken out of an assay container using tweezers.

Reverse Transcriptase-Loop-Mediated Isothermal Amplification (RT-LAMP) Reaction

The upper chamber of an assay container can comprise one or more set of primers for the amplification of one or more target nucleic acid molecules and a reverse transcriptase for a reverse transcriptase-loop-mediated isothermal amplification (RT-LAMP) reaction.

Loop-mediated isothermal amplification (LAMP) is a nucleic acid amplification technique that can be used for diagnosis and detection of disease-causing organisms. Typical LAMP assay can be performed using at least one set of primers comprising two inner and two outer primers that recognize six distinct regions of the target nucleic acid molecule (F1, F2 and F3 and B1, B2 and B3, respectively). The forward inner primer (FIP) consists of a F2 region and a complementary sequence of an F1 (Flo) region, while the backward inner primer (BIP) consists of the B2 region and a complementary sequence of the B1 (B1c) region. The forward outer primer (F3) and backward outer primer (B3) have sequences that are complementary to the sequences of the F3c and B3c regions, respectively. These regions surround the desired amplified sequence. The primers used for LAMP assays can be optimized by a series of factors, such as the nucleotide base pair concentration and locations, the distance between the DNA regions, the thermodynamics of the primers, etc.

A LAMP amplification process starts with the FIP complexing with a target DNA at its F2 region to form double-stranded DNA, in equilibrium at around 65° C. The DNA polymerase with strand displacement activity then initiates DNA synthesis from the FIP, simultaneously displacing a single strand of DNA, if any. After this initiation step, the F3 primer then binds to its complementary F3c region and displaces the FIP-complementary strand. Because of the F1c sequence in the FIP, the FIP strand is able to self-anneal and form a loop structure at one end of the DNA. This strand then serves as the target for BIP-initiated DNA synthesis and subsequent strand displacement from B3-primed DNA synthesis. This allows the other end of the single DNA strand to form a loop structure, thus resulting in a dumbbell-like DNA structure. It serves as the template for subsequent amplification. After a dumbbell-like structure formed, exponential amplification of the dumbbell structure is then initiated, with the DNA polymerase starting DNA synthesis at the F1 region. FIP also hybridizes to the one-loop structure from the F2 region, and DNA synthesis of that primer cause the F1-primed strand to be displaced and self-bind into a loop structure. Finally, from the B1 region in the new loop, self-primed DNA synthesis is again started, amplifying the current template while also creating a new one from the displacement of FIP-complementary strand. From this repeating process, it is possible for massive amplification to be achieved, as DNA can be amplified as much as 10⁹ times within an hour. To further improve the amplification efficiency, six-primer (loop primer) LAMP can be used. In LAMP with loop primers, six primers are used instead of four primers, with the forward and backward loop primers (LF and LB) annealing to regions between the F1/F2 and B1/B2 regions, respectively. The loop primers can enhance the LAMP process, as amplification is faster due to the increased starting points for DNA synthesis.

To extend LAMP from DNA diagnosis to RNA diagnosis as well as to realize multiplexed detection, several improvements were added to the original LAMP protocol to develop different methods, including reverse transcription LAMP (RT-LAMP) and multiplex LAMP. In RT-LAMP, RNA sequences are detected instead of DNA. Reverse transcriptase is added to the LAMP mixture to help in the conversion of RNA into complementary DNA (cDNA) that can be used for amplification. Multiplexed LAMP assays can detect multiple pathogens in the same test tube, with the use of more primers or with unique fluorescent signals.

A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses.

Retroviral RT has three sequential biochemical activities: RNA-dependent DNA polymerase activity, ribonuclease H (RNAse H), and DNA-dependent DNA polymerase activity. Collectively, these activities enable the enzyme to convert single-stranded RNA into double-stranded cDNA. In retroviruses and retrotransposons, this cDNA can then integrate into the host genome, from which new RNA copies can be made via host-cell transcription. The same sequence of reactions is widely used to convert RNA to DNA for use in molecular cloning, RNA sequencing, polymerase chain reaction (PCR), or genome analysis. Prime editor and genome editing in general rely on the use of RTases for their ability to generate DNA using an RNA template.

In an embodiment, the heat-stable or thermostable reverse transcriptase used for the RT-LAMP reaction can be an in-silico designed Moloney Murine Leukemia Virus (MMLV) reverse transcriptase variant or an in-silico designed avian myeloblastosis virus (AMV) reverse transcriptase variant. MMLV reverse transcriptase is known for its ability to synthesize DNA from a single-stranded RNA template.

RT-LAMP Primers

One or more sets of primers in the upper chamber can comprise two or more oligonucleotides. For example, the upper chamber can comprise 1, 2, 3, 4 or more sets of primers for the simultaneous amplification of 1, 2, 3, 4 or more target nucleic acid molecules. Each set of primers can comprise two or more oligonucleotides. For example, each set of primers can comprise 2, 3, 4, 5, 6 or more oligonucleotides. In an embodiment, a set of primers can comprise 4 oligonucleotides, for example a F3, a B3, a FIP and a BIP primers. In another embodiment, a set of primer can comprise 6 oligonucleotides, for example a F3, a B3, a FIP, a BIP, a LF and a LB primers.

RT-LAMP primer molecules are not particularly limited. As shown in the working examples, one of ordinary skill in the art can design sets of RT-LAMP primer molecules that are specific for any target nucleic acid molecule sequence. In fact, several tools are available to generate RT-LAMP primer molecules given a target nucleic acid molecule sequence (e.g., NEB® LAMP Primer Design Tool (Rowley MA, USA); PrimerExplorer (Eiken Chemical Co. LTD, Tokyo, Japan). Therefore, once a target nucleic acid molecule sequence is known, one of ordinary skill in the art can generate RT-LAMP primer molecules.

As used herein, polynucleotides can be used to refer to nucleic acid molecules comprising deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated, oligonucleotides (primers) or combinations thereof. Polynucleotides can be present as a single-stranded or double-stranded and linear or covalently circularly closed molecule.

Polynucleotides can also be synthesized in the laboratory, for example, using an automatic synthesizer. An amplification method such as PCR or LAMP can be used to amplify polynucleotides from either genomic DNA, RNA or cDNA encoding the polypeptides.

Polynucleotides can be isolated. An isolated polynucleotide can be a naturally occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally occurring genome is removed or absent. Isolated polynucleotides also include non-naturally occurring nucleic acid molecules. Polynucleotides can encode full-length polypeptides, polypeptide fragments, and variant or fusion polypeptides. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

A polynucleotide can comprise, for example, a gene, open reading frame, non-coding region, or regulatory element. A gene is any polynucleotide molecule that encodes a polypeptide, protein, or fragment thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide.

The term “polynucleotide” can refer to one or more polypeptides (e.g., a set of oligonucleotides or primers).

Oligonucleotides can be designed to amplify any target nucleic acid molecule, that is any prokaryotic, eukaryotic, viral, or synthetic nucleic acid molecule. A nucleic acid molecule can be disease-causing or be a nucleic acid of an organism that is disease-causing. A target nucleic acid molecule can comprise a sequence coding a gene product (e.g., a protein) or a fragment thereof, or a non-coding sequence (e.g., a regulatory polynucleotide or a non-coding DNA). Once a target nucleic acid molecule is identified, primers for the specific amplification of said target nucleic acid molecule can be designed. Many tools are available for the design of primers based on the sequence of a target nucleic acid molecule. For example, the Primer-Blast tool of the National Center for Biotechnology Information (NCBI) can be used (ncbi.nlm.nih.gov/tools/primer-blast).

Primers can hybridize to a target nucleic acid with high specificity, that is a change in one or more nucleotides in a target nucleic acid can prevent the hybridization (and therefore amplification) of said target nucleic acid molecule. For the same reasons, the design of primers can be done such that target nucleic acid molecules comprising nucleotide changes can be specifically recognized and amplified. For example, nucleotide changes can include single nucleotide polymorphism (SNP). That is, primers can be designed specifically to hybridize to and to amplify SNP, thereby detecting the presence in a sample of specific variants of a target nucleic acid molecule.

The two or more oligonucleotides in the upper chamber of the assay container can comprise, for example, SEQ ID NOs:1-6 or SEQ ID NOs:7-12. Such oligonucleotides can be used for the amplification of two different target nucleic acid molecules (N gene and E gene) of a SARS-CoV-2 virus.

For example, an upper chamber of the assay container can comprise one set of 4 primers, for the amplification of one target nucleic acid molecule, such as SEQ ID NOs:1-4, or SEQ ID NOs:7-10. In another example, the upper chamber of the assay container can comprise one set of 6 primers, for the amplification of one target nucleic acid molecule, such as SEQ ID NOs:1-6 or SEQ ID NOs:7-12.

An upper chamber of an assay container can comprise two sets of 4 primers, for the simultaneous amplification of two target nucleic acid molecules, such as SEQ ID NOs:1-4 and SEQ ID NOs:7-10. In another example, the upper chamber of the assay container can comprise two sets of 6 primers, for the simultaneous amplification of two target nucleic acid molecules, such as SEQ ID NOs:1-6 and SEQ ID NOs:7-12.

TABLE 1
Oligonucleotide primers' names
and sequences

| Oligonucleotide | | |
|---|---|---|
| Primers | | SEQ ID |
| Name | Sequence (5′ → 3′) | NO: |
| F3 2019-nCoV | AACACAAGCTTTCGGCAG | SEQ ID |
| N-gene | | NO: 1 |
| B3 2019-nCoV | GAAATTTGGATCTTTGTC | SEQ ID |
| N-gene | ATCC | NO: 2 |
| BIP 2019-nCoV | TGCGGCCAATGTTTGTAA | SEQ ID |
| N-gene | TCAGCCAAGGAAATTTTG | NO: 3 |
| | GGGAC | |
| FIP 2019-nCoV | CGCATTGGCATGGAAGTC | SEQ ID |
| N-gene | ACTTTGATGGCACCTGTG | NO: 4 |
| | TAG | |
| LF 2019-nCoV | TTCCTTGTCTGATTAGTTC | SEQ ID |
| N-gene | | NO: 5 |
| LB 2019-nCoV | ACCTTCGGGAACGTGGTT | SEQ ID |
| N-gene | | NO: 6 |
| F3 2019-nCoV | CCGACGACGACTACTAGC | SEQ ID |
| E-gene | | NO: 7 |
| B3 2019-nCoV | AGAGTAAACGTAAAAAGA | SEQ ID |
| E-gene | AGGTT | NO: 8 |
| BIP 2019-nCoV | ACCTGTCTCTTCCGAAAC | SEQ ID |
| E-gene | GAATTTGTAAGCACAAGC | NO: 9 |
| | TGATG | |
| FIP 2019-nCoV | CTAGCCATCCTTACTGCG | SEQ ID |
| E-gene | CTACTCACGTTAACAATA | NO: 10 |
| | TTGCA | |
| LF 2019-nCoV | TCGATTGTGTGCGTACTG | SEQ ID |
| E-gene | C | NO: 11 |
| LB 2019-nCoV | TGAGTACATAAGTTCGTA | SEQ ID |
| E-gene | C | NO: 12 |

Hyperthermophilic Archaeon Argonaute Protein-Based Target Sequence Detection Reaction

The lower chamber of an assay container can comprise a hyperthermophilic archaeon Argonaute protein, one or more sets of single-stranded guide DNA (gDNA) specific for one or more target nucleic acid molecules, and one or more reporter molecules for a hyperthermophilic archaeon Argonaute protein-based target sequence detection reaction.

Ago Protein

Hyperthermophilic archaeon Argonaute proteins can be e.g., from Pyrococcus furiosus, Thermococcus thioreducens (WP 055429304), Thermococcus onnurineus (WP 012572468), Thermococcus eurythermalis (WP_050002102), Thermus thermophilus, Methanocaldococcus bathoardescens (WP_048201370), Methanocaldococcus sp. FS406-22 (WP_012979970), Methanocaldococcus fervens (WP 015791216), Methanocaldococcus jannaschii (WP_010870838), Methanotorris formiscicus (WP 052322764), Ferroglobus placidus (WP_012966655), Sulfolobus sp. (e.g., S. solfataricus), Methanopyrus kandleri, and Thermogladius cellulolyticus (WP 048163021).

In an embodiment, an Ago protein has 25, 30, 40, 50, 60, 70, 80, 90, 95, 99 percent or more sequence identity to an Pyrococcus furiosus Ago protein. In an embodiment an Ago protein is from thermophilic or hyperthermic bacteria or archaea. Thermophilic bacteria or archaea have growths temperatures of about 40° C. to about or more and an optimal growth temperature of about 60° C. Hyperthermic bacteria or archaea have growth temperatures of about 65° C. to 120° C. and an optimal growth temperature of about 80° C.

Ago proteins utilize small DNA or RNA guides to cleave single-stranded DNA targets. PfAgo protein from Pyrococcus furiosus is a DNA-guided nuclease (771 amino acids) that targets cognate DNA.

In an embodiment, a coding sequence encoding an Ago protein, e.g., PfAgo, can be codon optimized for expression in certain host cells, such as prokaryotic or eukaryotic cells. Prokaryotic cells include, for example E. coli. Eukaryotic cells can be for example, yeast cells or mammalian cells such as human, mouse, rat, rabbit, dog, or non-human primate. Codon optimization is a process of modifying a nucleic acid sequence for enhanced expression in host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Different species can exhibit a bias for certain codons of an amino acid. Codon bias (differences in codon usage between organisms) can correlates with efficiency of translation of messenger RNA (mRNA). Therefore, Ago polynucleotides can be altered for optimal gene expression in a given organism based on codon optimization. Codon usage tables are known in the art. See Nakamura et al. “Codon usage tabulated from the international DNA sequence databases: status for the year 2000” Nucl. Acids Res. 28:292 (2000). Computer algorithms for codon optimizing a particular sequence for expression in a particular host cell are also available, e.g., Gene Forge (Aptagen; Jacobus, Pa.). In an embodiment, one or more codons (e.g. 1, 2, 3, 4, 5, 10, 15, 20, 50, or more, or all codons) in a nucleotide sequence encoding an Ago protein correspond to the most frequently used codon for a particular amino acid in a host cell.

Single-Stranded Guide DNA (gDNA)

A lower chamber of an assay container can comprise one or more sets of single-stranded guide DNA (gDNA) specific for one or more target nucleic acid molecules.

A “DNA guide target sequence” is a sequence within a target DNA to which a DNA guide sequence is designed to have complementarity, where hybridization between a target nucleic acid molecule and a DNA guide sequence/Ago complex promotes the formation of an Ago complex (e.g., a complex of one or more DNA guides, one or more Ago proteins, and a target nucleic acid molecule). Full complementarity between a DNA guide molecule and a DNA guide target sequence is not necessarily required, provided there is sufficient complementarity to cause hybridization and promote formation of an Ago complex.

Typically, in the context of an Ago system, formation of an Ago complex results in cleavage of the DNA target molecule within or near (e.g. within 1, 2, 3, 4, 5, 6, 7, 8, 9, 10 or more base pairs from) the DNA guide target sequence.

Systems can comprise one or more single-stranded DNA guides. One or more of the single-stranded DNA guides in a system can be 5′-phosphorylated, 3′-phosphorylated or both. One or more DNA guides can target two strands of DNA (sense and antisense strand) at any desired locations and cleave the strands in separate events. Once each strand of DNA is cleaved at the desired location, the two strands can re-anneal or hybridize to generate desired cleaved dsDNA.

A guide molecule is any single-stranded deoxyribonucleic acid molecule having sufficient complementarity with a target deoxyribonucleic acid molecule to hybridize with the target molecule. A guide molecule forms a complex with an Ago protein and directs sequence-specific binding of the guide molecule/Ago complex to the target DNA molecule. The degree of complementarity between a guide molecule and its corresponding target DNA molecule, when optimally aligned using a suitable alignment algorithm, is about 50%, 60%, 75%, 80%, 85%, 90%, 91%, 92%, 93%, 94%, 95%, 96%, 97%, 98%, 99%, or more. In an embodiment the complementarity is 100%. Optimal alignment can be determined with any suitable algorithm for aligning sequences, for example, Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, and ELAND (Illumine, San Diego, Calif.). A guide sequence can be about or more than 9, 11, 12, 13, 14, 15, 16, 17, 18, 19, 20, 21, 22, 23, 24, 25, 26, 27, 28, 29, 30, 35, 40, 50, 55, 60, 65, 70, 75, 80, 90, 100 or more nucleotides in length (or any range between about 9 and 100 nucleotides). A guide sequence can be less than about 100, 80, 75, 60, 50, 45, 40, 35, 30, 29, 28, 27, 26, 25, 24, 23, 22, 21, 20, 19, 18, 17, 16, 14, 13, 12, 11, or 10 nucleotides in length.

The ability of a guide molecule to direct sequence-specific binding of Ago to a target DNA molecule can be tested using any suitable assay. For example, cleavage of a target DNA molecule can be evaluated in vitro by contacting a test target molecule, Ago, one or more test guide molecules having homology to the test target molecule, and a control guide sequence different from the test guide sequence, and comparing binding and/or rate of cleavage of the target DNA molecule between the test guide and control guide sequence reactions.

A guide molecule can be selected or designed to target and hybridize to any target molecule at a DNA guide target sequence. DNA guide target sequences can be unique in the target DNA molecule or can occur at two or more locations with the target DNA molecule, In an embodiment, an Ago protein can be used in combination with one or more single-stranded DNA guide sequences, e.g., 1, 2, 3, 4, 5, 6, 7, 8, 9, 10, 11, 12, 15, 20, 25, 30, or more guide sequences that target sense and/or antisense strands of target DNA molecules. This system allows both target DNA strands of the double-stranded DNA molecule to be nicked. Alternatively, only one target DNA strand can be nicked.

A DNA guide or guide DNA (gDNA) hybridizes to a DNA guide target sequence on a sense strand of a target molecule. A target sequence or target nucleic acid molecule can be any prokaryotic, eukaryotic, viral, or synthetic nucleic acid molecule, that is sought to be detected using the methods described herein. As such, in some instances, e.g., when the target nucleic acid is a viral or a synthetic nucleic acid molecule, the target nucleic acid can be an RNA and not a DNA molecule. In such cases, a target nucleic acid molecule (e.g., a viral RNA) can be reverse transcribed into DNA prior to being contacted with a DNA guide to ensure that the DNA guide can hybridize to the target nucleic acid molecule.

In some embodiments, a guide molecule is designed to reduce the degree of secondary structure within the guide molecule. Secondary structure can be determined by any suitable polynucleotide folding algorithm including, for example mFold (Zuker and Stiegler (Nucleic Acids Res 9 (1981), 133-148)).

In an embodiment, the guide molecules are designed so that a first guide molecule hybridizes to a sense strand of a denatured or partially denatured DNA molecule and a second guide molecule hybridizes to an anti-sense strand of a denatured or partially denatured DNA molecule. The first and second guide molecules can have the same sequence or, more often, different sequences. The first and second guide molecules can be designed so that when the Ago protein cleaves the sense and antisense strand of the target DNA the target molecule has overlapping or sticky ends (ends of DNA that can hybridize to one another). In another embodiment, the guide molecules are designed so that when the Ago protein cleaves the sense and antisense strand of the target DNA, blunt ends are generated.

gDNA molecules are not particularly limited. As shown in the working examples, one of ordinary skill in the art can design sets of gDNA molecules that are specific for any target nucleic acid molecule sequence. In fact, several tools are available to generate gDNA molecules given a target nucleic acid molecule sequence (e.g., GenScript®, developed by the Broad Institute of Harvard and MIT; CHOPCHOP (Labun, K., Montague, T. G., Krause, M., Torres Cleuren, Y. N., Tjeldnes, H., & Valen, E. CHOPCHOP v3: expanding the CRISPR web toolbox beyond genome editing. Nucleic Acids Research (2019)). Therefore, once a target nucleic acid molecule sequence is known, one of ordinary skill in the art can generate gDNA molecules.

In an embodiment, the Ago protein, e.g., PfAgo protein, and the one or more single-stranded DNA guides do not occur together in nature. In an embodiment, single-stranded DNA guides are synthetic and do not occur in nature. In an embodiment, the single-stranded DNA guides are not from Pyrococcus furiosus or Thermus thermophilus. In an embodiment the single-stranded DNA guides comprise one or more labels or tags.

A DNA guide molecule and Ago protein form a complex. The guide molecule provides target specificity to the complex by comprising a nucleotide sequence that is complementary to a sequence of a target DNA. The guide of the complex provides the site-specific activity. In other words, the Ago protein is guided to the target DNA molecule (e.g., a viral sequence, a chromosomal sequence or an extrachromosomal sequence, e.g. an episomal sequence, a minicircle sequence, a mitochondrial sequence, a chloroplast sequence, etc.) by virtue of its association with at least the DNA guide molecule. The Ago protein cleaves target DNA. In some cases, the Ago is a naturally occurring polypeptide. In other cases, the Ago is not a naturally occurring polypeptide (e.g., a chimeric polypeptide or a naturally occurring polypeptide that is modified by, e.g., amino acid mutation, deletion, or insertion).

In an embodiment, a DNA guide molecule hybridizes to a DNA guide target sequence on a sense strand of a target molecule within about 1, 2, 3, 4, 5, 10, 15, 20, 30, 40, 50, 100, 200, 300, 400, 500, 1,000 nucleotides or more of where a DNA guide molecule hybridizes to a DNA guide target sequence on an anti-sense strand of the target DNA molecule (determined from prior to denaturation or partial denaturation of the target DNA molecule).

The lower chamber of the assay container can comprise one or more sets of gDNA specific for one or more target nucleic acid molecules. For example, the lower chamber of the assay container can comprise 1, 2, 3, 4 or more sets of gDNA for the simultaneous detection of 1, 2, 3, 4 or more target nucleic acid molecules. Each set of gDNA can comprise two or more gDNAs. For example, each set can comprise 2, 3, 4, 5, 6 or more gDNAs. The two or more gDNAs in the lower chamber of the assay container can comprise two or more of e.g., SEQ ID NOs:13-15 or SEQ ID NOs:16-18.

For example, the lower chamber of the assay container can comprise one set of 2 gDNAs, for the detection of one target nucleic acid molecule, such as SEQ ID NOs:13-14, 13-15, 14-15, 16-17, 16-18, or 17-18. In another example, the upper chamber of the assay container can comprise one set of 3 gDNAs, for the detection of one target nucleic acid molecule, such as SEQ ID NOs:13-15 or SEQ ID NOs:16-18.

The lower chamber of the assay container can comprise two set of 2 gDNAs, for the detection of two target nucleic acid molecules, such as SEQ ID NOs:13-14 and 16-17, 16-18, or 17-18, SEQ ID NOs:13-15 and 16-17, 16-18, or 17-18, or SEQ ID NOs: 14-15 and 16-17, 16-18, or 17-18. In another example, the lower chamber of the assay container can comprise two set of 3 gDNAs, for the detection of two target nucleic acid molecules, such as SEQ ID NOs:13-15 and SEQ ID NOs:16-18.

TABLE 2
gDNAs' names and sequences

| Guide DNAs | | |
|---|---|---|
| (gDNAs) | Sequence | SEQ ID |
| Name | (5′ → 3′) | NO: |
| SARS-CoV-2 | /5Phos/ATGCGCGA | SEQ ID |
| N gDNA-1 | CATTCCGA/3Phos/ | NO: 13 |
| SARS-CoV-2 | /5Phos/AGAACGCT | SEQ ID |
| N gDNA-2 | GAAGCGCT/3Phos/ | NO: 14 |
| SARS-CoV-2 | /5Phos/CCCCAGCG | SEQ ID |
| N gDNA-3 | CTTCAGCG/3Phos/ | NO: 15 |
| SARS-CoV-2 | /5Phos/TGGCTAGT | SEQ ID |
| E gDNA-1 | GTAACTAG/3Phos/ | NO: 16 |
| SARS-CoV-2 | /5Phos/TGGCTAGT | SEQ ID |
| E gDNA-2 | GTAACTAG/3Phos/ | NO: 17 |
| SARS-CoV-2 | /5Phos/TTGCTTTC | SEQ ID |
| E gDNA-3 | GTGGTATT/3Phos/ | NO: 18 |

Reporter Molecules

The lower chamber of the assay container can comprise one or more reporter molecules. A reporter molecule or probe can comprise a nucleic acid molecule having complementarity to a target nucleic acid molecule, for the specific interaction of the reporter molecule with the amplified target nucleic acid molecules, and a fluorescent moiety, for the fluorescent detection of the amplified target nucleic acid molecules. A fluorescent moiety can be attached or linked to a nucleic acid molecule. The sets of gDNAs can induce targeted Ago cleavage on the amplicon to release a ssDNA fragment, which acts as a secondary gDNA, targeting Ago cleavage to the specific Fluorophore-Quencher (F-Q) labeled ssDNA reporter probe. A sequence-specific cleavage of a quenched probe can link a unique secondary gDNA to the release of a fluorescent moiety. A secondary gDNA can be designed to have an optimal length, thereby improving the detection of a fluorescent signal, and reducing the number of false positives. For example, a secondary gDNA can comprise 14, 15, 16, 17, 18 or more nucleotides. In an embodiment, a secondary gDNA can comprise 16 nucleotides.

Cleaved single-stranded DNAs resulting from the Argonaute protein-based target sequence detection reaction can subsequently be hybridized to generate desired cleaved double-stranded DNA (dsDNA). For example, the cleaved single-stranded DNAs can be hybridized with a reporter molecule, such as a nucleic acid molecule having complementarity to the one or more target nucleic acid molecules comprising a fluorescent moiety, for the detection of the cleaved dsDNA.

The lower chamber of the assay container can comprise one or more reporter molecules for the specific detection of one or more target nucleic acid molecules. For example, the lower chamber of the assay container can comprise 1, 2, 3, 4 or more reporter molecules for the detection of 1, 2, 3, 4 or more target nucleic acid molecules.

In an embodiment, the lower chamber can comprise one reporter molecule comprising a nucleic acid molecule having complementarity to the target nucleic acid molecule and having SEQ ID NO:19 or SEQ ID NO:20.

In another embodiment, the lower chamber can comprise two reporter molecules comprising a nucleic acid molecule having complementarity to the target nucleic acid molecules and having SEQ ID NO:19 and SEQ ID NO:20.

TABLE 3
Reporter molecules' name and sequences

| Reporter Molecules | | SEQ ID |
|---|---|---|
| Name | Sequence (5' > 3') | NO: |
| SARS-COV-2 N | /56-FAM/TTCCGAAG | SEQ ID |
| reporter probe | AACGCTGAA/3BHQ_1/ | NO: 19 |
| SARS-COV-2 E | /56-ROXN/AACTAGC | SEQ ID |
| reporter probe | AAGAATACCA/3BHQ_2/ | NO: 20 |

A reporter molecule can comprise a fluorescent moiety. Any fluorescent dye that can be attached or linked to a nucleic acid molecule can be used as a fluorescent moiety. For example, the fluorescent moiety can be a xanthene derivative (such as fluorescein, rhodamine, Oregon green, eosin, and Texas red), a cyanine derivative (such as cyanine, indocarbocyanine, oxacarbocyanine, thiacarbocyanine, and merocyanine), a squaraine derivative or ring-substituted squaraines (such as Seta and Square dyes), a squaraine rotaxane derivative, a naphthalene derivative (such as dansyl and prodan derivatives), a coumarin derivative, an oxadiazole derivative (such as pyridyloxazole, nitrobenzoxadiazole and benzoxadiazole), an anthracene derivative (such as anthraquinones, including DRAQ5, DRAQ7 and CyTRAK Orange), a pyrene derivative (such as cascade blue), an oxazine derivatives (such as Nile red, Nile blue, cresyl violet, oxazine 170, etc.), an acridine derivative (such as proflavin, acridine orange, acridine yellow, etc.), an arylmethine derivative (such as auramine, crystal violet, malachite green), a tetrapyrrole derivative (such as porphin, phthalocyanine, bilirubin) or a dipyrromethene derivative (such as BODIPY, aza-BODIPY).

In one embodiment, a fluorescent moiety can be carboxyfluorescein (FAM) or carboxyrhodamine (ROX).

6-Carboxyfluorescein (6-FAM) and carboxyrhodamine are fluorescent dyes with an absorption wavelength of 495 nm and 525 nm, respectively. A carboxyfluorescein molecule is a fluorescein molecule with a carboxyl group added. Rhodamine is a family of related dyes, a subset of the triarylmethane dyes derivatives of xanthene, and including carboxyrhodamine. They are both commonly used as a tracer agent.

Target Nucleic Acid Molecules

As used herein, a target nucleic acid molecule can be any prokaryotic, eukaryotic, viral or synthetic nucleic acid molecule. A target nucleic acid molecule can comprise a sequence coding a gene product (e.g., a protein) or a non-coding sequence (e.g., a regulatory polynucleotide or a non-coding DNA). A target nucleic acid molecule can be a polynucleotide that comprises a target site or nucleic acid guide target sequence. The terms target site or target sequence refer to a nucleic acid sequence present in a target nucleic acid to which a DNA guide and/or a oligonucleotide can hybridize to. Suitable DNA binding conditions include physiological conditions normally present in a cell. Other suitable DNA binding conditions are known in the art; see, e.g., Green & Sambrook, Molecular Cloning: A Laboratory Manual (Fourth Edition), Cold Spring Harbor Laboratory Press (2012).

The target nucleic acid molecule described herein can include bacterial nucleic acid molecule, viral nucleic acid molecule, fungal nucleic acid molecule, eukaryote nucleic acid molecule, or nucleic acid molecule from any source that one would want to detect.

For example, a target nucleic acid can be a viral nucleic acid molecule, such as a nucleic acid molecule from an RNA virus. An RNA virus is a virus that has RNA (ribonucleic acid) as its genetic material. This nucleic acid can single-stranded RNA (ssRNA) or double-stranded RNA (dsRNA). Non-limiting examples of human diseases caused by RNA viruses include common cold (caused by rhinoviruses), influenza (caused by influenza viruses), SARS (caused by SARS-CoV coronavirus), MERS (caused by MERS coronavirus), COVID-19 (caused by SARS-CoV-19 coronavirus), dengue fever (cause by dengue virus), hepatitis C (caused by hepatitis C virus), hepatitis E (caused by hepatitis E virus), West Nile fever (caused by West Nile virus), Ebola hemorrhagic fever (caused by Ebolaviruses), rabies (caused by rabies virus and Australian bat lyssavirus), poliomyelitis (caused by poliovirus) and measles (caused by measles virus). All of the sequences of these viruses are available at, for example, Gen Bank®. In such cases, a target nucleic acid molecule (e.g., the viral RNA) can be reverse transcribed into DNA prior to being contacted with a DNA guide to ensure that the DNA guide can hybridize to the target nucleic acid molecule.

In an embodiment, the one or more target nucleic acid molecules can be one or more SARS-CoV-2 nucleic acid molecules.

Severe acute respiratory syndrome coronavirus 2 (SARS-CoV-2) is the virus that causes coronavirus disease 2019 (COVID-19), the respiratory illness responsible for the COVID-19 pandemic. SARS-CoV-2 is a positive-sense single-stranded RNA virus (that is contagious in humans). As described by the US National Institutes of Health, it is the successor to SARS-CoV-1, the strain that caused the 2002-2004 SARS outbreak. Taxonomically, SARS-CoV-2 is a strain of severe acute respiratory syndrome-related coronavirus (SAR-Sr-CoV).

Each SARS-CoV-2 virion is 50-200 nanometres in diameter. Like other coronaviruses, SARS-CoV-2 has four structural proteins, known as the S (spike, accession number NC_045512.2), E (envelope, accession number NC_045512.2), M (membrane, accession number NC_045512.2), and N (nucleocapsid, accession number NC_045512.2) proteins; the N protein holds the RNA genome, and the S, E, and M proteins together create the viral envelope. The spike protein, which has been imaged at the atomic level using cryogenic electron microscopy, is the protein responsible for allowing the virus to attach to and fuse with the membrane of a host cell; specifically, its S1 subunit catalyzes attachment, the S2 subunit fusion.

In an embodiment, the SARS-CoV-2 nucleic acid molecule can be an envelope (E) nucleic acid molecule, a nucleoprotein (N) nucleic acid molecule, a spike (S) nucleic acid molecule, a membrane (M) nucleic acid molecule, or a combination thereof.

Sets of Containers

Provided herein is a set of containers comprising: a sample preparation container and an assay container described herein.

An assay container described herein can be used in association with one or more additional containers. For example, a sample, such as a saliva sample, can be collected from a subject using a collection device and transferred into a sample preparation container, the prepared sample can be transferred into the assay container using a transfer container.

As used herein, a “collection device” can be any suitable device that can be used to collect a given sample from a subject, such as a saliva sample. Non-limiting examples of suitable collection device include a collection capillary, a syringe, a pipette, and a swab.

As used herein, a “sample preparation container” can be any container that can contain a sample and a DNA extraction solution. A sample preparation container can include a generally tubular body, or any other suitable shape, with an open end. A sample preparation container can include an aperture running lengthwise of the container that can accommodate a DNA extraction solution. For example, the sample preparation container can be a tube, a well, or a chip. The sample preparation container can be made out of any material that is compatible with the conditions (e.g., temperature conditions) required to perform DNA extraction. For example, the assay container can be made out of polypropylene, metal, glass, or any other suitable solid, nonabsorbent material, and can be partially or fully translucent for visibility. For example, the container can be a capillary tube.

As used herein, a “DNA extraction solution” can comprise the elements required to extract DNA from the sample, such as a detergent or surfactant solution to induce the lysis of the cells present in the sample, and a salt to separate the nucleic acid molecules from the proteins associated with it. Optionally, a DNA extraction solution can comprise an alcohol, such as ethanol, to induce the precipitation of the nucleic acid molecules.

As used herein, a “transfer container” can be any container that can be used to remove the prepared sample (e.g., after completion of the DNA extraction reaction) from the sample preparation container, and to insert said prepared sample into the assay container. Non-limiting examples of transfer container include a collection capillary tube, a syringe, and a pipette.

Portable Diagnostic Device

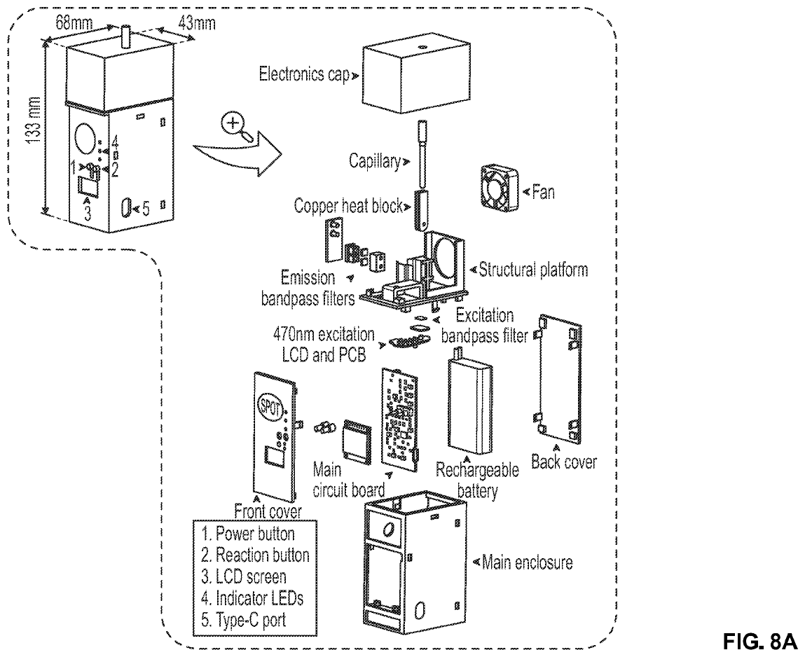

Provided herein is a portable diagnostic device comprising a) a heating module, b) a fluorometry module, c) the assay container described herein within an opening to hold the assay container, and d) an LCD screen, see e.g., FIG. 8A.

A diagnostic device is designed to host one or more assay container as described herein, wherein a RT-LAMP reaction, and a hyperthermophilic archaeon Argonaute protein-based target sequence detection reaction are to occur. The device is thus designed to include a heating module comprising a copper heat block wrapped with nichrome wire and an integrated fan to adjust the temperature to the reaction being performed. The copper heat block can be configured to allow temperature control, and the integrated fan can be configured to enable cooling.

The fluorometry module can comprise an LED board, a photodiode board, emission bandpass filters, and a motherboard comprising a microcontroller. The emission bandpass filters can be mounted perpendicular to the capillary and filter light to the photodiodes. The LED board can comprise a blue LED located beneath the excitation bandpass filter, the blue LED can shine light into a hole at the bottom of the copper heat block and into the assay container. The blue LED can have e.g., a 470 nm excitation. Any wavelength that will excite both fluorescent probes (FAM and ROX) can be suitable. For example, both fluorophores, can be excited to some degree by most of the ultraviolet, violet, and blue spectrum (e.g. about 100, 200, 300, 380, 400, or 500 nm (or any range between about 100 and 500 nm). The excitation LED can be filtered by a 470 nm maximum with 20 nm full width at half maximum (FWHM) bandpass filters. One or more bandpass filters can be used for the detection of one or more fluorescent moieties, such that several fluorescent moieties with different absorption/emission profile can be detected using a same fluorometry module. For example, for quantification of ROX fluorescence, a 610 nm maximum 20 nm FWHM bandpass filter can be used. For quantification of FAM fluorescence, a 520 nm maximum 20 nm FWHM bandpass filter can be used.

The device can further comprise a transimpedance amplifier configured to convert photodiode current into voltage, which can be read by the microcontroller. The photodiode board can comprise one or more photodiodes secured behind the emission bandpass filters.

The device can further comprise a rechargeable and removable lithium-ion battery. For the portability of the device, a rechargeable and removable lithium-ion battery can be used to provide the power required for the device to work. Alternatively, the device can be wired to allow for a non-portable use of the device.

The device is designed so that an excitation light shines through the sample to ensure the specific and accurate detection of the amplified target nucleic acid molecules via detection of fluorescent moieties. To avoid the rise of false-negative or false-positive results, that could be the result of an inappropriate light exposition, the device can further comprise a cap covering the device to block excess light.

A device can be designed following the same description as detailed herein, with for example a larger heating system, and several separated modules for fluorescent quantification. That is, a device can be designed to allow for several assay container to be inserted into the device such that several samples can be tested at a time. Such enlarge system can improve the number of samples tested. For example, using such a device, 250, 500, 750, 1000, 1250, 1500 or more samples can be tested per day.

Methods of Use

Provided herein are methods of detecting one or more target nucleic acid molecules in a sample comprising a) amplifying one or more target nucleic acid molecules using a RT-LAMP reaction to produce amplified target nucleic acid molecules, b) contacting the amplified target nucleic acid molecules with a hyperthermophilic archaeon Argonaute protein, one or more sets of gDNA specific for the one or more target nucleic acid molecules, and one or more reporter molecules, and c) detecting fluorescence in the sample.

The methods described herein can comprise several steps that can be performed individually, each in a different assay container, or successively in a single assay container.

In one embodiment, steps a), b) and c) can be performed in a single assay container. In such conditions, the assay container can be designed to allow the RT-LAMP reaction to occur first, in conditions that are suitable for an RT-LAMP amplification reaction to produce amplified target nucleic acid molecules. In a second step, a hyperthermophilic archaeon Argonaute protein-based target sequence detection reaction occurs, which detects and cleaves the amplified target nucleic acid molecules. For example, steps a), b) and c) can be performed in an assay container comprising a removable barrier separating an upper chamber, comprising one or more sets of primers for the amplification of one or more target nucleic acid molecules for a RT-LAMP reaction, from a lower chamber comprising a hyperthermophilic archaeon Argonaute protein, one or more sets of gDNA specific for one or more target nucleic acid molecules, and one or more reporter molecules for a hyperthermophilic archaeon Argonaute protein-based target sequence detection reaction.

In a RT-LAMP amplification reaction, a sample can be contacted with the one or more sets of primers specific for amplification of the target nucleic acid molecules and a reverse transcriptase, and the sample can be heated at temperature suitable to perform the RT-LAMP reaction.

Target nucleic acid molecules can be one or more bacterial nucleic acid molecules, viral nucleic acid molecules, fungal nucleic acid molecules, eukaryote nucleic acid molecules, or nucleic acid molecules from any source that one would want to detect. In one embodiment, the one or more target nucleic acid molecules can be SARS-CoV-2 nucleic acid molecules. For example, the one or more SARS-CoV-2 nucleic acid molecules can be envelope (E) nucleic acid molecules, nucleoprotein (N) nucleic acid molecules, spike (S) nucleic acid molecules, membrane (M) nucleic acid molecules, or a combination thereof.

The one or more sets of primers can comprise 2 or more oligonucleotides designed specifically for the amplification of the one or more target nucleic acid molecules of interest. For example, the one or more sets of primers can comprise 2, 3, 4, 5, 6, 7, 8 or more oligonucleotides for the amplification of SARS-CoV-2 E nucleic acid molecules or N nucleic acid molecules. The one or more sets of primers can comprise 2 sets of 6 oligonucleotides selected from SEQ ID NOs:1-6 and SEQ ID NOs:7-12.

The sample can be heated at a temperature ranging from about 60° C. to about 65° C., or at any temperature suitable for the optimal activity of the RT selected for the RT-LAMP reaction. For example, the sample can be heated at 58, 59, 60, 61, 62, 63, 64, 65, 66, 67, 68° C. or more. The sample can be heated for 5 or more minutes, such that enough target nucleic acid molecules can be amplified. The sample can be heated for, e.g., 5, 10, 15, 20, 25, 30, 35, 40, 45 or more minutes. In one embodiment, the sample is heated at 65° C. for about 20-30 minutes.

A removable barrier can be removed from an assay container at the end of an RT-LAMP reaction to ensure that the amplified nucleic acids molecules can be contacted with the elements in the lower chamber for an Argonaute protein-based target sequence detection reaction to occur. A removable barrier can be a meltable plug or a solid physical barrier, such as a plastic, a silicone or a rubber plug. When a removable barrier is a meltable plug, a meltable plus can be removed from an assay container by heat, that is by heating an assay container at a temperature that is higher than the melting point of the meltable plug, a meltable plus can dissolve resulting in the removal of the barrier. When a removable barrier is a solid physical barrier, a barrier can be physically removed from an assay container. For example, a solid physical barrier can be taken out of an assay container using tweezers.

During the Argonaute protein-based target sequence detection reaction, the amplified target nucleic acid molecules can be contacted with one or more sets of gDNA specific for the one or more target nucleic acid molecules, and with one or more reporter molecules, and the sample can be heated at a temperature suitable to perform the Argonaute protein-based target sequence detection reaction. When target nucleic acid molecules are viral nucleic acid molecules, target nucleic acid molecules can be RNA target nucleic acid molecules and not DNA target nucleic acid molecules. In such case, the target nucleic acid molecules can be reverse transcribed into DNA prior to being contacted with a DNA guide to ensure that the DNA guide can hybridize to the target nucleic acid molecule.

The one or more sets of gDNA can comprise 2 or more gDNAs selected and designed specifically for the detection and cleavage of the amplified target nucleic acid molecules. For example, the one or more sets of gDNA can comprise 2, 3, 4, 5 or more gDNAs for the detection and cleavage of SARS-CoV-2 E nucleic acid molecules or N nucleic acid molecules. The one or more sets of gDNA can comprise 2 sets of 3 gDNAs selected from SEQ ID NOs:13-15 and SEQ ID NOs:16-18.

The sample can be heated at a temperature ranging from about 90° C. to about 100° C. or at any temperature suitable for the optimal activity of the Argonaute protein. For example, the sample can be heated at 85, 86, 87, 88, 89, 90, 91, 92, 93, 94, 95, 96, 97, 98, 99, 100, 101, 102, 103° C. or more. The sample can be heated for about 3 minutes. For example, the sample can be heated for 1, 2, 3, 4, 5, 6, 7 or more minutes. In one embodiment, the sample is heated at 90° C. for about 3 minutes.

The one or more reporter molecules can comprise a nucleic acid molecule having complementarity to the target nucleic acid molecules and a fluorescent moiety. The nucleic acid molecules having complementarity to the target nucleic acid molecules can comprise SEQ ID NO:19 or SEQ ID NO:20. The fluorescent moiety can be carboxyfluorescein (FAM) or carboxyrhodamine (ROX).

Provided herein are methods of diagnosing an RNA virus infection in a subject. For example, the RNA virus infection can be a COVID-19 infection, caused by SARS-CoV-2 coronavirus. Examples of other RNA viruses are orthomyxoviruses, Hepatitis A virus, Hepatitis B Virus, Hepatitis C Virus, Ebola, SARS, MERS, influenza, polio, measles, and retroviruses including adult Human T-cell lymphotropic virus type 1 (HTLV-1) and human immunodeficiency virus (HIV). The method can comprise detecting one or more RNA virus (e.g., SARS-CoV-2 virus) nucleic acid molecules in a sample from the subject comprising: a) loading the sample into an upper chamber of a assay container comprising a removable barrier separating an upper chamber from a lower chamber, the upper chamber comprising one or more set of primers specific for the amplification of one or more RNA virus (e.g., SARS-CoV-2 virus) nucleic acid molecules and the reverse transcriptase for a RT-LAMP reaction and the lower chamber comprising a hyperthermophilic archaeon Argonaute protein, one or more sets of gDNA specific for one or more RNA virus (e.g., SARS-CoV-2 virus) nucleic acid molecules, and one or more reporter molecules for a hyperthermophilic archaeon Argonaute protein-based target sequence detection reaction, b) inserting the assay container into a portable diagnostic device comprising: a heating module, an fluorometry module, an opening to insert and hold the assay container, and an LCD screen, c) heating the sample at 60° C. to 65° C. for 5 minutes or more; d) heating the sample at 90° C. to 100° C. for 3 minutes or more; and e) reading a diagnostic test result on the LCD screen.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

As used herein “diagnosing an RNA virus infection”, such as a COVID-19 infection for example, refers to the detection of one or more target nucleic acid molecules from the RNA virus (e.g., SARS-CoV-2 virus) in a sample collected from the subject. As detailed below, when an RNA virus (e.g., SARS-CoV-2 virus) nucleic acid molecules are detected in a sample collected from a subject, this can indicate that the subject is currently infected by said RNA virus (e.g., SARS-CoV-2 virus), and therefore is positive for an RNA virus infection (e.g., COVID-19 infection). However, when an RNA virus (e.g., SARS-CoV-2 virus) nucleic acid molecules are not detected in a sample collected from a subject, this can indicate that the subject is not currently infected by said RNA virus (e.g., SARS-CoV-2 virus), and therefore is negative for an RNA virus infection (e.g., COVID-19 infection). A negative result is only an indication of a patient's status regarding the RNA virus infection (e.g., COVID-19 infection) at the time the sample was collected from the subject. It does not indicate if the subject has been previously infected and does not indicate that a subject is not at risk of developing an RNA virus infection (e.g., a COVID-19 infection) at a later time.

COVID-19 infection is a disease caused by SARS-CoV-2 coronavirus, that is responsible for COVID-19 pandemic. The detection of a COVID-19 infection in a subject by the methods described herein can further comprise the administration of a treatment to the subject to lessen one or more symptoms of a COVID-19 infection. For example, an antiviral such as remdesivir can be administered to reduce viral replication; steroid medication, such as dexamethasone can be administered to reduce overactive immune response; monoclonal antibodies such as bamlaniviman, casirivimab and imdevimab can be administered to help the immune system recognize end fight against the infection; and acetaminophen or ibuprofen can be administered to reduce symptoms such as aches and fever. Additional treatment may also be administered to treat COVID-19 infection complications, such as heart, blood vessels, kidneys, brain, skin, eyes and gastrointestinal complications. For example, blood thinners can be administered to prevent or treat blood clots.

The sample can be loaded into an upper chamber of a assay container comprising a removable barrier separating an upper chamber from a lower chamber. The sample can be for example a saliva sample. In some aspects, the sample can be prepared prior to being loaded in the upper chamber of the assay container. For example, the sample can be collected from a subject using a collection device (such as a collection capillary, a syringe, a pipette or a swab), and transferred into a sample preparation container. A sample collection container can comprise a nucleic acids extraction solution, to extract DNA and/or RNA from the sample upon heating of the preparation container (and sample). The sample comprising extracted nucleic acids can then be transferred into the upper chamber of an assay container.

The upper chamber of the assay container can comprise one or more set of primers specific for the amplification of one or more RNA viruses (e.g., SARS-CoV-2 virus) nucleic acid molecules and the reverse transcriptase for a RT-LAMP reaction. For example, the upper chamber of the assay container can comprise one or more sets of primers comprising six oligonucleotides selected from SEQ ID NOs:1-6 and SEQ ID NOs:7-12. In one embodiment, the upper chamber comprises two sets of 6 oligonucleotides selected from SEQ ID NOs:1-6 and SEQ ID NOs:7-12.

At this point, the assay container can be inserted into a portable diagnostic device comprising: a heating module, a fluorometry module, an opening to insert and hold the assay container, and an LCD screen, as described herein. The device can be turned on, which can lead to the sample being heated at 60° C. to 65° C. for 5 minutes or more, at a temperature and during a time suitable for the amplification of one or more RNA viruses (e.g., SARS-CoV-2 virus) nucleic acid molecules by the reverse transcriptase. For example, the sample can be heated at 65° C. for 20-30 minutes.

The portable device can be programmed to increase the temperature to about 90° C. to 100° C. at the end of the time set to be required for the RT-LAMP reaction. Alternatively, after the RT-LAMP amplification has run for a period of time evaluated by a user to be sufficient to amplify one or more RNA viruses (e.g., SARS-CoV-2 virus) nucleic acid molecules, a user can manually increase the temperature of the heating module to about 90° C. to 100° C. The increase in the temperature can be used as a mean to remove the removable barrier of the assay container. For example, if the removable barrier is a meltable plug, by increasing the temperature to about 90° C. to 100° C., the meltable plug can be transformed from a solid state to a melted state. Alternatively, if a solid removable barrier is used, a user can manually remove the physical barrier, for example using tweezers. The removal of the removable barrier can allow the content of the upper chamber (e.g., the amplified material from the sample) to enter in contact with the content of the lower chamber. At this point, the amplified nucleic acid molecules can be contacted with a hyperthermophilic archaeon Argonaute protein, one or more sets of gDNA specific for one or more RNA viruses (e.g., SARS-CoV-2 virus) nucleic acid molecules, and one or more reporter molecules such that a hyperthermophilic archaeon Argonaute protein-based target sequence detection reaction can occur.

The lower chamber of the assay container can comprise one or more sets of gDNA. For example, the lower chamber of the assay container can comprise one or more sets of gDNA comprising three gDNAs selected from SEQ ID NOs:13-15 and SEQ ID NOs:16-18. In one embodiment, the lower chamber comprises two sets of three gDNAs selected from SEQ ID NOs:13-15 and SEQ ID NOs:16-18.

The lower chamber of the assay container can comprise one or more reporter molecules. For example, the lower chamber can comprise two reporter molecules comprising a nucleic acid molecule having complementarity to the RNA virus (e.g., SARS-CoV-2 virus) nucleic acid molecules and a fluorescent moiety. The nucleic acid molecule having complementarity to the RNA virus (e.g., SARS-CoV-2 virus) nucleic acid molecule can comprise SEQ ID NO:19 or SEQ ID NO:20. The fluorescent moiety can be carboxyfluorescein (FAM) or carboxyrhodamine (ROX). In one embodiment, the first reporter molecule can comprise a nucleic acid molecule having complementarity to the SARS-CoV-2 nucleic acid molecule comprising SEQ ID NO:19 and a FAM fluorescent moiety, and the second reporter molecule can comprise a nucleic acid molecule having complementarity to the SARS-CoV-2 nucleic acid molecule comprising SEQ ID NO:20 and a ROX fluorescent moiety.

At this point, the assay container (still inserted into the portable diagnostic device) can be heated at about 90° C. to 100° C., which can lead the sample to being heated at 90° C. to 100° C. for about 3 minutes, at a temperature and during a time suitable for Argonaute protein-based target sequence detection by the hyperthermophilic archaeon Argonaute protein. For example, the sample can be heated at 90° C. for 3 minutes.

The portable device can be programmed to automatically cool down to a lower temperature, such as room temperature for example, at the end both reactions. At this point, if fluorescence is detected by the fluorometry module, it can indicate that one or more RNA viruses (e.g., SARS-CoV-2 virus) nucleic acid molecules were present in the sample, amplified during the RT-LAMP reaction, and detected during the Argonaute protein-based target sequence detection reaction. The detection of fluorescence in the sample can thus indicate a positive RNA virus (e.g., SARS-CoV-2 virus) infectious status of the subject. In such a case, the portable device can be programmed to automatically display the result on the LCD screen. For example, “positive,” “+,” or the like can be displayed on the LCD screen.

If no fluorescence is detected by the fluorometry module, it can indicate that no RNA virus (e.g., SARS-CoV-2 virus) nucleic acid molecules were present in the sample. The absence of detection of fluorescence in the sample can thus indicate a negative RNA virus (e.g., SARS-CoV-2 virus) infectious status of the subject. In such a case, the portable device can be programmed to automatically display the result on the LCD screen. For example, “negative,” “-,” or the like can be displayed on the LCD screen.

The methods described herein allows for a sensitive detection and quantification of RNA virus (e.g., SARS-CoV-2 virus) nucleic acid molecules in a sample. The methods described herein can be used to detect about less than about 500, 250, 100, 60, 50, 40, 30, 20 or less copies of RNA virus nucleic acid molecules in a sample. That is, the diagnostic device described herein can provide a positive test result if a minimum of about 500, 250, 100, 60, 50, 40, 30, 20 or less copies of RNA virus nucleic acid molecules are detected in a sample.

For example, a limit of detection of SARS-CoV-2 N nucleic acid can be established at about 17.5 copies, that is the methods described herein can detect and provide a positive test result if a minimum of about 17.5 copies of a SARS-CoV-2 N nucleic acid molecules are detected in the sample (after amplification). Additionally, a limit of detection of SARS-CoV-2 E nucleic acid can be established at about 43.8 copies, that is the methods described herein can detect and provide a positive test result if a minimum of about 43.8 copies of a SARS-CoV-2 N nucleic acid molecules are detected in the sample (after amplification).

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1. Methods

Specimen and Nucleic Acid Preparation

Three types of specimens were tested using the SPOT assay:
A. In-vitro transcribed (IVT) RNA: Synthesized DNA fragments of SARS-CoV-2 were used to generate IVT templates. A PCR step was performed on the synthetic gene fragment with a forward primer containing a T7 promoter. Next, the PCR product was used as the template for an IVT reaction at 37° C. for 4 hours using Precision RNA Synthesis Kit (A29377, Invitrogen). The IVT reaction was then treated with DNase I for 15 min at 37° C. followed by an RNA purification step. The RNA was quantified by Nanodrop and Qubit and spiked into pooled human saliva (IRHUSL5ML, Innovative Research Inc.) to the specified concentration.
B. Gamma-irradiated SARS-CoV-2 (NR-52287, BEI Resources): Irradiated virus samples were spiked into pooled human saliva and treated with QuickExtract DNA Extraction Solution (QE09050, Lucigen) at 95° C. for 5 min with the 1:1 ratio.
C. Clinical saliva samples: The clinical saliva samples, obtained from the Veterinary Diagnostic Laboratory of University of Illinois at Urbana-Champaign, were treated with QuickExtract DNA Extraction Solution (QE09050, Lucigen) at 95° C. for 5 min at a 1:1 ratio.

Design of Reporter Probe and gDNAs

The reporter probe will generate a quantifiable fluorescent output signal. For the detection of SARS-CoV-2 using the SPOT assay, we used a 17 nt reporter probe for PfAgo recognition and cleavage. Dual phosphorylated 16 nt gDNAs were used for primary cleavage to prevent false priming during the LAMP reaction. The SPOT assay combines LAMP amplification with PfAgo detection. For LAMP amplification, two sets of primers (total 6 oligos) were used to target the SARS-CoV-2 N (nucleoprotein) and E (envelope) genes respectively.

SPOT Assay

Reagents:
Bst 2.0 WarmStart® DNA Polymerase (M0538L), New England Biolabs (NEB) (Ipswich, MA)
WarmStart® RTx Reverse Transcriptase (M0380L), NEB
Magnesium Sulfate (MgSO4) Solution (B1003S), NEB
Isothermal Amplification Buffer Pack (B0537S), NEB
Deoxynucleotide (dNTP) Solution Mix (N0447S), NEB
PfAgo protein was purified as described protocol 10, 11, stored as 30 μL aliquots at 5 mg/mL
LAMP primers for amplifying N gene and E gene can be ordered from Integrated DNA Technologies (IDT) (Coralville, IA).
gDNAs for detecting N gene and E gene can be ordered from IDT.
Reporter DNA for fluorescence readout (N gene: /56-FAM/XXX/3BHQ-1/, E gene: /6-ROX/XXX/3BHQ-2/), were ordered from IDT.
50 mM MnCl2 (100×), prepared by nuclease-free water.

TABLE 4
10× LAMP primer mix:

| LAMP Primer | |
|---|---|
| (100 μM, in | Amount |
| nuclease-free water) | (μL) |
| F3 | 2 |
| B3 | 2 |
| FIP | 16 |
| BIP | 16 |
| LF | 8 |
| LB | 8 |
| Nuclease-free water | 48 |
| Total | 100 |

TABLE 5
SPOT assay mastermix:

| | Initial | | Final | | Amount |
|---|---|---|---|---|---|
| | concentration | | concentration | | (μL) |
| Upper chamber | | | | | |
| WarmStart ® Bst 2.0 | 8000 | units/mL | 320 | units/mL | 1.6 |
| WarmStart ® RTx | 15,000 | units/mL | 300 | units/ml | 0.8 |
| Isothermal amplification buffer | 10× | | 1× | | 4 |
| dNTPs | 10 | mM | 1.4 | mM | 5.6 |
| MgSO₄ | 100 | mm | 8 | mM | 3.2 |
| N gene primer mix | 10× | | 0.5× | | 2 |
| E gene primer mix | 10× | | 0.5× | | 2 |
| Saliva samples | | | | | 5 |
| Nuclease-free water | | | | | 15.8 |
| Total | | | | | 40 |
| Lower chamber | | | | | |
| PfAgo | 5 mg/ml or 55 μM | | 1.375 | μM | 2 |
| MnCl₂ | 50 | mM | 0.5 | mM | 0.8 |
| Isothermal amplification buffer | 10× | | 1× | | 4 |
| Non-primer oligos | 100 | μM | 625 | nM | 3 |
| (total 6 oligos) | | | | | |
| Reporter probe 1 | 100 | μM | 156.25 | nM | 0.125 |
| Reporter probe 2 | 100 | μM | 312.5 | nM | 0.25 |

| | |
|---|---|
| Nuclease-free water | 29.825 |
| Total | 40 |

SPOT Assay Performed in a Thermocycler

The LAMP reactions (upper chamber mixture) were performed in a thermocycler. After 20-30 min at 63° C., the target RNA was reverse-transcribed and amplified to generate enough DNA for detection. Then, the PfAgo components (lower chamber mixture) were added into LAMP products. As the temperature was raised to PfAgo initiated cleavage reaction and released the fluorescent signal. For samples containing SARS-CoV-2 nucleic acids, 3-5 min incubation at 95° C. generated quantifiable fluorescent output.

One-Step SPOT Protocol for SARS-CoV-2 Saliva Sample Detection

A. Preparation of Capillary 1 (for Saliva Pretreatment) and Capillary 2 (for SPOT Detection)
Capillary 1: 10 μL QuickExtract DNA Extraction Solution is loaded at the bottom of the capillary and sealed with a plastic stopper.
Capillary 2: 40 μL PfAgo compartment is loaded into the bottom of the capillary. Then, 20 mg paraffin wax powder is added into the capillary followed by heating the capillary above 70° C. for 1 minute for complete melting of the paraffin wax. After paraffin wax congeals, the 35 μL LAMP reaction is inserted into the capillary, on top of the paraffin wax.

Paraffin wax powder preparation: use a blade to cut some paraffin wax chips from the larger block, then place the chips into a mortar and add liquid nitrogen until it completely covers the chips. Grind chips gently in the mortar and collect the powder in a glass bottle.

B. Testing Procedure

Use collection capillary (1-000-0200, MICROCAPS®) to obtain around 10 μL of saliva from patient's mouth via automatic suction by the capillarity of tube.

Press the rubber cap to inject the saliva sample into capillary 1 and mix with the lysis buffer.

Insert capillary 1 into the SPOT device and press the reaction button to initiate program 1 (95° C. for 5 min).

Remove capillary 1 from the device and use a new collection capillary to transfer around 3 μL lysate into the upper compartment of capillary 2.

Insert capillary 2 into the SPOT device and press the reaction button to initiate program 2 (63° C. for 30 min, 95° C. for 5 min, followed by a 1-minute fan cooling period). For samples containing SARS-CoV-2 nucleic acids, 3 min incubation at 95° C. generates quantifiable fluorescent output.

The test readout will display on the screen of the device with “Positive” or “Negative”.

qRT-PCR Analysis of Clinical Saliva Samples

qRT-PCR analysis of clinical saliva samples was performed by the Veterinary Diagnostic Laboratory of University of Illinois at Urbana-Champaign, using the Fisher TaqPath COVID-19 Combo kit (Applied Biosystems) and QuantStudio 3 Real-Time PCR System (Applied Biosystems) according to a previously reported protocol 16.

Design and Assembly of the SPOT Device

The SPOT device is comprised of 3D printed structural parts, a machined copper heat block, optical filters, and electrical components. CAD files of 3D printed parts and the copper heat block can be found in the supplementary materials. Parts were printed using a Form 3 (Formlabs; Somerville, MA) 3D printer with Black Resin V4 (RS-F2-GPBK-04, Formlabs). The copper heat block was machined at the University of Illinois Aerospace and Physics-MRL machine shops. PCBs were fabricated by ALLPCB (Zhejiang, China). Optical filters were purchased from Salvo Technologies (Pinellas Park, Florida). The excitation LED was filtered by a 470 nm maximum with 20 nm full width at half maximum (FWHM) bandpass filter (SKU: C0674-91, Salvo Technologies). For quantification of ROX fluorescence, a 610 nm maximum 20 nm FWHM bandpass filter was used (SKU: C0674-65, Salvo Technologies). For quantification of FAM fluorescence, a 520 nm maximum 20 nm FWHM bandpass filter was used (SKU: 102387140, Salvo Technologies).

The motherboard and battery are mounted into the bottom enclosure, which has openings for the charging cable, USB cable, power and start buttons, an LCD screen, and indicator LEDs. The main platform fits on the top of the bottom enclosure and houses the thermal and optical modules. The optical module consists of a blue LED (710-150353BS74500, Mouser; Mansfield, TX) beneath the excitation bandpass filter which shines into a hole at the bottom of the copper heat block, directly into a capillary. Two emission bandpass filters are mounted perpendicular to the capillary and filter light passing to the photodiodes. A transimpedance amplifier is used to convert photodiode current into voltage to be read by the microcontroller. The user-selected gain is then used to configure the programmable-gain amplifier which results in increased dynamic range. The copper heat block is wrapped with fiberglass insulated nichrome wire and heats upon application of an electrical current. After completion of the reaction, an integrated fan turns on for rapid cooling. To block excess light, a cap covers the platform section containing a circular opening for insertion and removal of capillaries. The CAD file for the entire SPOT device assembly can be found in the supplementary materials.

Using the SPOT dashboard software, a computer can be used to monitor and configure the SPOT device, quantify sample fluorescence, and export data. With the configuration utility, the time and temperature for the LAMP and PfAgo cleavage reactions can be modified if desired, which by default are set to 63° C. for 30 minutes and 95° C. for 5 minutes, respectively. User guides for the SPOT device and dashboard software can be found in the supplementary information.

Statistical Analysis

Data is shown as original data or the mean with error bars representing standard deviations. Graphs were made by using the GraphPad Prism software (version 9.0).

Code Availability

The custom codes used in this study, including but not limited to the SPOT device microcontroller, display, fluorescent quantification, and data analysis, are available from the corresponding author upon request. Codes can also be accessed on GitHub following this link (gitlab.engr.illinois.edu/ispot).

### Example 2. System for COVID-19 Diagnosis

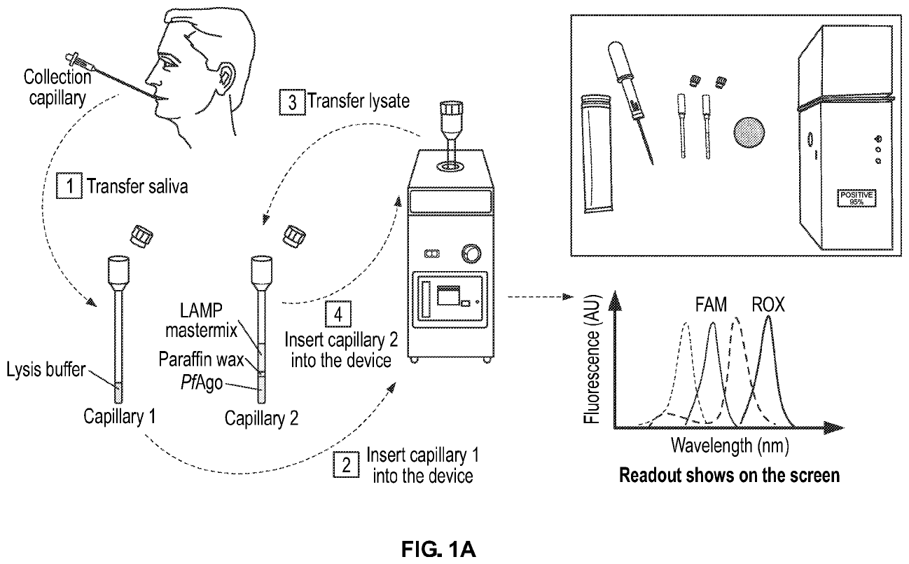

The workflow of the SPOT system is shown in FIG. 1A. Briefly, pre-treated saliva samples can be tested automatically in a portable device which includes precise heating and optical modules, producing quantifiable fluorescent output signals upon detection of SARS-CoV-2 nucleic acid sequences. Initially, the saliva sample collected by a capillary can be mixed with QuickExtract DNA extraction solution in a sample preparation capillary (capillary 1) for heat pre-treatment. Using a transfer capillary, users inject pretreated saliva samples into the assay capillary (capillary 2) containing reagents for viral RNA amplification and detection. Detection can then be initiated by switching on the device: the heating module can first hold at 63° C. for 20-30 min for LAMP amplification followed by automatically heating to 95° C. for 3-5 min for PfAgo detection, and finally the LED excitation module can shine through the capillary. Fluorescence is detected by photodiodes secured behind bandpass filters specific for emissions of the chosen fluorophores and quantitative test results are displayed on an attached LCD screen.

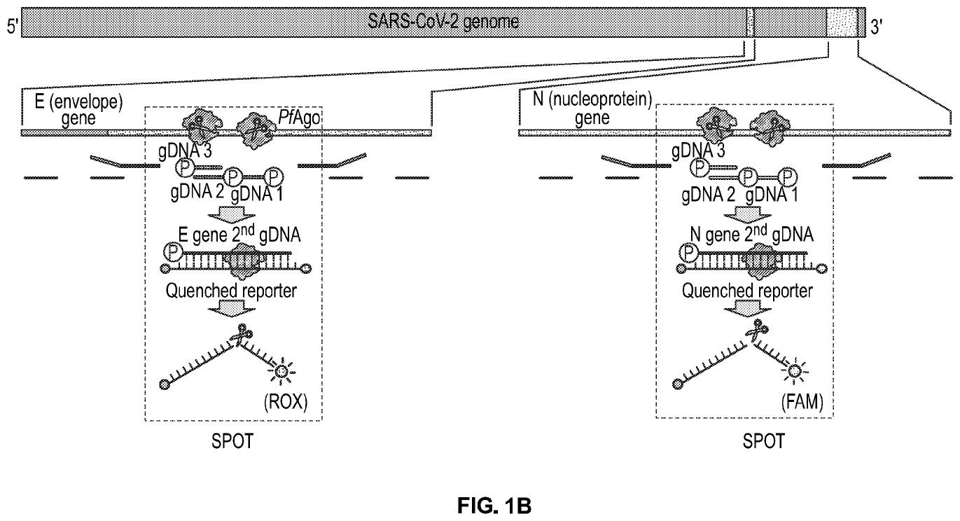

To develop the assay for COVID-19 diagnostics using the SPOT system, RT-LAMP was used to amplify the E (envelope) and N (nucleoprotein) genes of SARS-CoV-2. A set of gDNAs induce targeted PfAgo cleavage on the amplicon to release a 16 nucleotide (nt) ssDNA fragment, which acts as a secondary gDNA, targeting PfAgo cleavage to the specific Fluorophore-Quencher (F-Q) labeled ssDNA reporter probe (FIG. 1B). Since the reagents may affect the structure of the reporter and overall fluorescent background signal, the GC content and length of the reporter was optimized to minimize the unwanted background fluorescence. It was found that 17 nt reporters was the minimum size necessary to enable secondary cleavage, with low G-C content and labeled with a 5′-fluorophore and 3′-quencher produced relatively low background signals. The cleavage efficiency was compared in target gene detection reactions containing two or three primary gDNAs and it was found use of three gDNAs dramatically enhanced the efficiency of cleavage, which led to a higher fluorescent signal than use of two gDNAs (FIG. 2C).

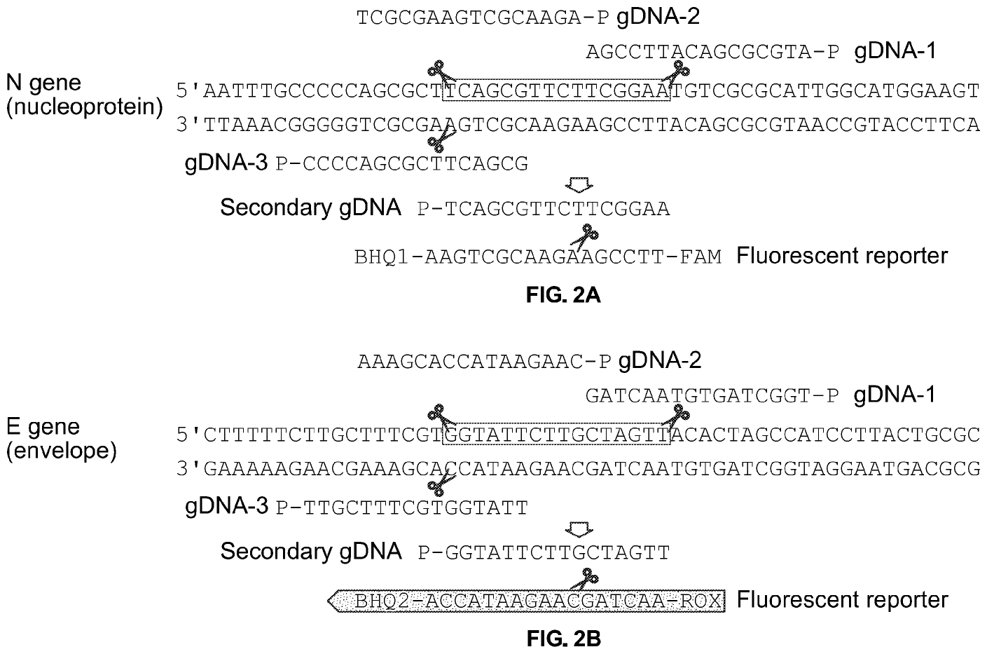

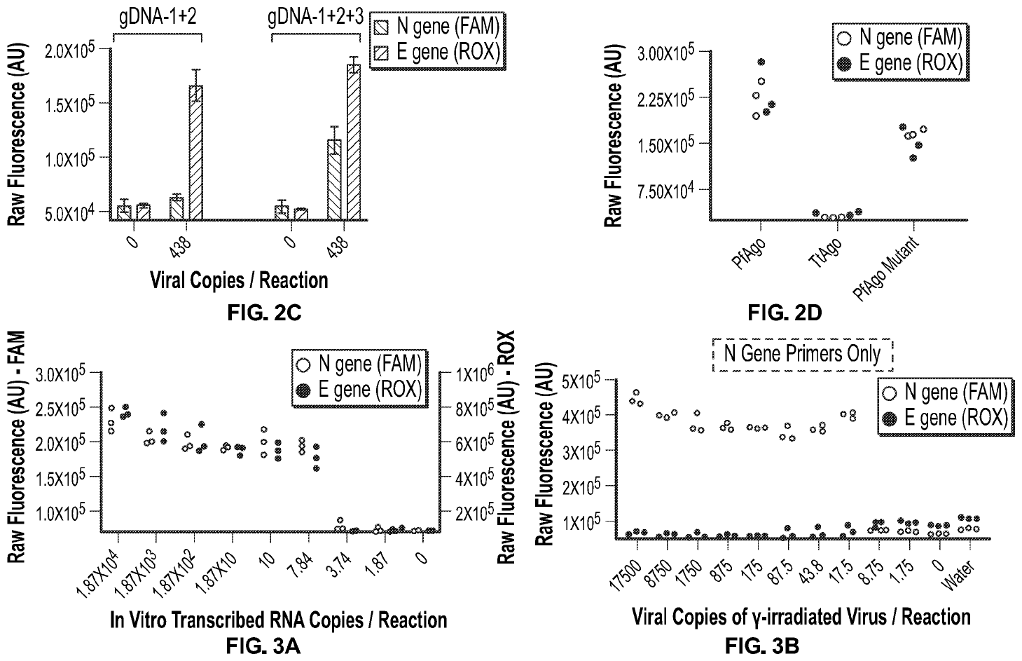

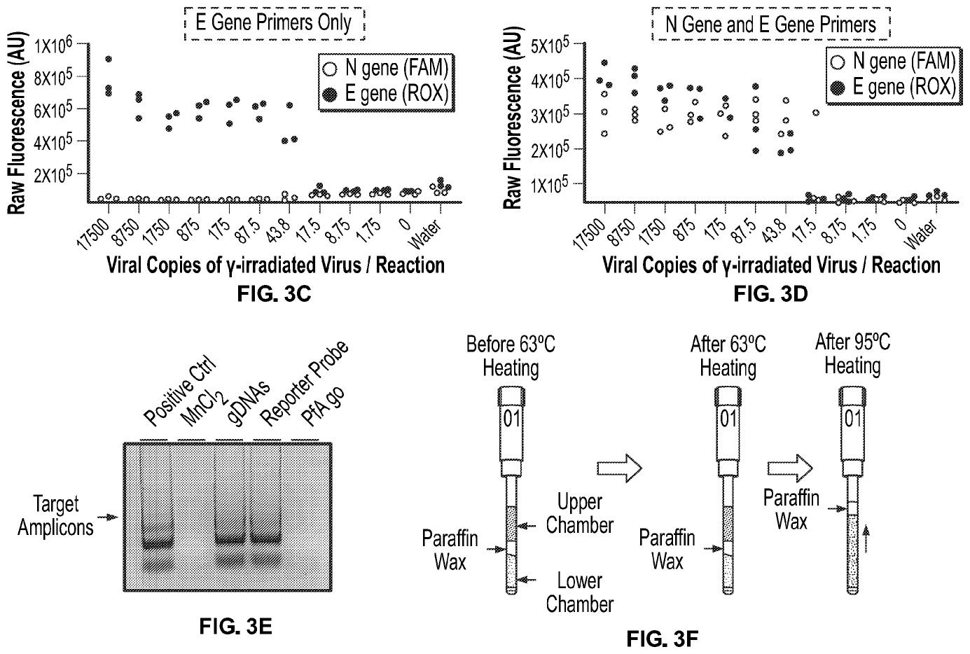

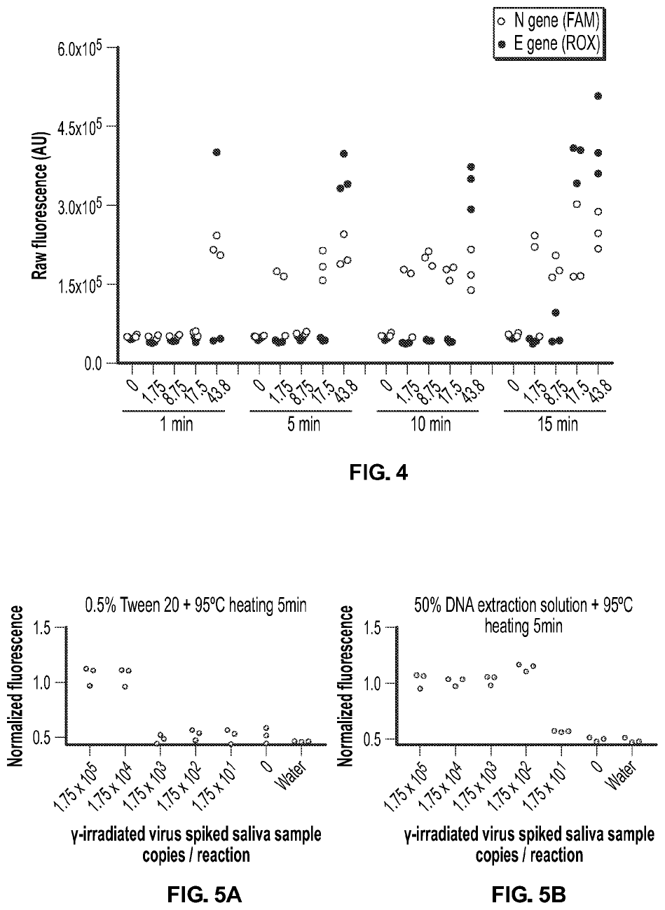

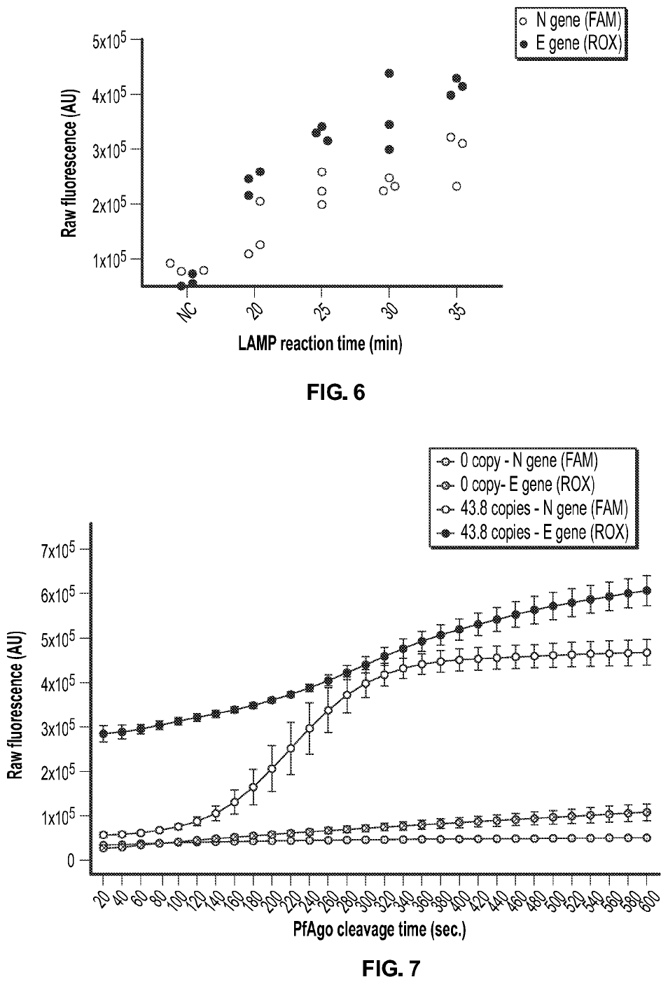

the limit of detection (LOD) of the SPOT assay by spiking saliva samples was determined with synthetic in vitro-transcribed (IVT) SARS-CoV-2 RNA followed by RT-LAMP and PfAgo-based detection reactions. After 20 min LAMP amplification at 63° C., the PfAgo detection components were added into the reaction mixture followed by heating at 95° C. for 3 min and quantification of readouts with a fluorometer. It was found that the LOD of the SPOT assay is ˜7.5 copies/reaction (0.19 copies/μL) with in vitro transcribed RNA samples (FIG. 3A). To determine the feasibility of the assay for detection of SARS-CoV-2 in real saliva samples, saliva samples spiked with gamma-irradiated SARS-CoV-2 were assayed. The virus-spiked saliva samples were first pre-treated by adding QuickExtract DNA Extraction Solution and heating at 95° C. for 5 min (FIG. 4 and FIG. 5A-B). The LOD was as low as 17.5 copies per reaction (0.44 copies/μL). Extending the LAMP reaction to 30 min can be used to detect SARS-CoV-2 in samples with an original viral number below 50 copies per reaction (FIG. 6). Using a time-course assay, we found that a minimum of 5 min is sufficient to obtain the maximum fluorescent output signal for detecting contrived saliva samples with the LOD (FIG. 7).

To minimize false positives and achieve high accuracy, two viral genes were detected simultaneously in the same reaction. two sets of gDNAs targeting the N and E genes with two different fluorophores were designed to establish a one-pot multiplexed reaction. When the N or E gene related primers were added into reaction separately, the specific fluorescence was only observed, accordingly, showing that PfAgo mediated multiplexed reaction is carried out in an orthogonal manner. Our method can detect the N and E genes in a multiplexed reaction with a LOD of 17.5 copies (FIG. 3B) and 43.8 copies (FIG. 3C), respectively, using virus-spiked saliva samples. We then added two sets of primers in one reaction, and the result showed that both two viral genes can be amplified and detected successfully (FIG. 3D).

To perform one-pot amplification and detection, some optimizations are required as components of the LAMP and PfAgo detection reactions are incompatible. The 5′-3′ dual-phosphorylated primary gDNAs used to mediate primary cleavage prevent priming during LAMP. Additionally, manganese ions (Mn²⁺), which are key to PfAgo cleavage activity, inhibit the LAMP reaction and PfAgo itself can partially inhibit amplification by tightly binding to DNA (FIG. 3E). Thus, an encapsulation method was developed to isolate the LAMP and PfAgo reaction components. The PfAgo reaction mixture was loaded into the capillary (diameter=3.18 mm), sealed with paraffin wax, followed by loading the LAMP reaction mixture to the upper chamber of the capillary. After heating above 70° C., the paraffin wax melts, releasing the PfAgo reaction mixture and initiating the detection process (FIG. 3F).

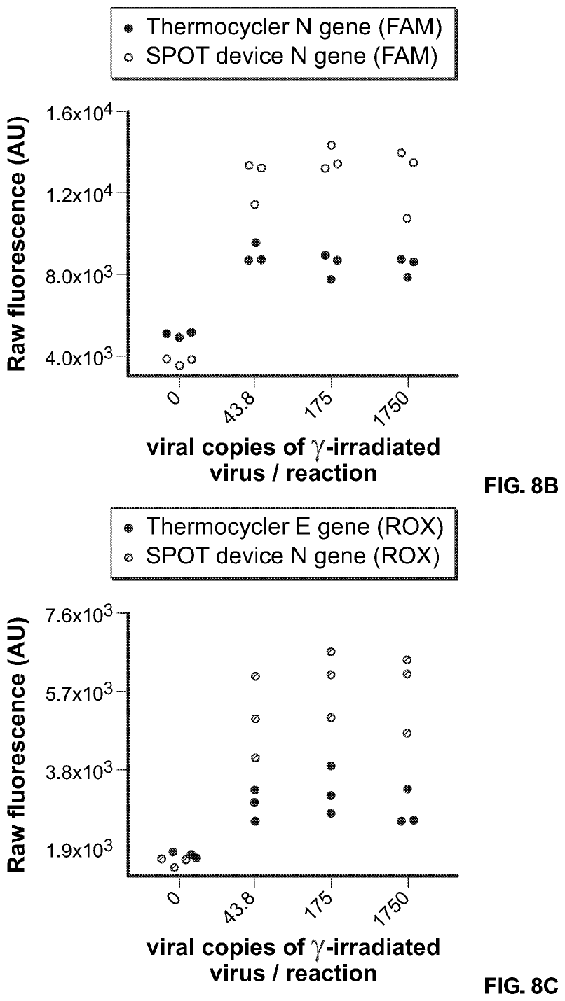

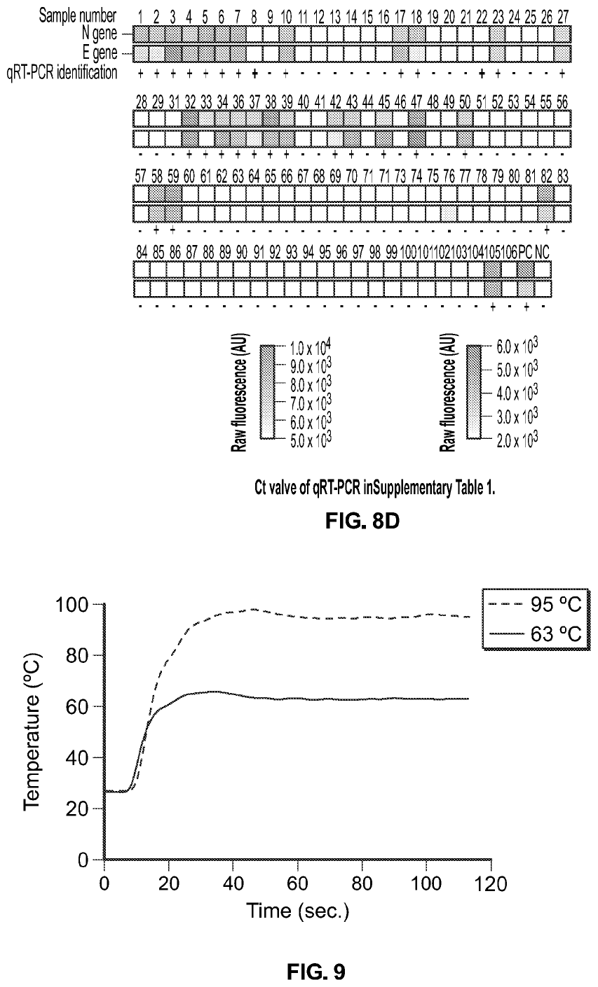

Next, a prototype portable device capable of performing sample pretreatment was designed and built, the assay, and quantifying the fluorescent output (FIG. 8A). This prototype device consists of 3D printed casing and internal structure with temperature control accomplished via a copper heat block wrapped with nichrome wire. An integrated fan enables rapid cooling of the sample capillary after completion of pretreatment or detection reactions. Fluorescent excitation is accomplished using a single blue LED aimed into the bottom of the capillary with fluorescent emissions detected by photodiodes placed behind fluorophore-specific emission filters. Temperature is controlled and fluorescence is quantified by three custom printed circuit boards: an LED board, a photodiode board, and a motherboard equipped with a microcontroller. The front of the device contains buttons for powering the device, starting or resetting the assay, and an LCD screen to display results. The system is powered by a rechargeable and swappable lithium-ion battery. This assembled prototype was named as SPOT device and directly compared with commercial machines in its ability to perform temperature control and fluorescence quantification. To evaluate the accuracy and stability of its heating module, reactions were conducted using both the SPOT device and a thermocycler. The fluorescence readouts obtained from the SPOT device and a qPCR machine were compared (QuantStudio 3 Real-Time PCR system), demonstrating that the SPOT device is as capable as a commercial thermocycler at performing the SPOT assay (FIGS. 8B and 8C, FIG. 9). Moreover, a comparison of end-point fluorescence readouts from the SPOT device and a qPCR machine showed that the SPOT device is sensitive enough to accurately replicate the fluorescent measurements obtained from the qPCR machine (FIG. 10).

Finally, the performance of the SPOT system was evaluated in a blinded test using 104 clinical saliva samples obtained from the Veterinary Diagnostic Laboratory at the University of Illinois at Urbana-Champaign. These samples were analyzed independently by scientists not involved in this study using the qRT-PCR approach and three target genes: ORF1ab gene (encoding a replication protein), S gene (encoding the spike protein), and N gene (encoding the nucleoprotein). By comparison, we were able to reliably detect 28 out of 30 (93.3%) SARS-CoV-2 positive patient samples (both N and E genes were detected) and 72 out of 74 (97.3%) SARS-CoV-2 negative samples (neither N nor E genes were detected). Two samples were declared inconclusive as only one of the two target genes was detected (see Table 6). After re-testing, one of these inconclusive samples was found to be negative but the other remained inconclusive (FIG. 8D). The two missed positive samples each had a Ct value of above 31 cycles for N gene and 32.3-36.6 cycles for S and ORF1ab genes. These Ct values correspond to original sample viral copy numbers of 5×10²-2.5×10³ copies/mL, which are below the LOD of the SPOT system when using saliva samples. However, when using in vitro transcribed RNA samples, the LOD of the SPOT system matches the LOD of the CDC's gold standard qRT-PCR testing system, indicating that optimization of the sample pretreatment method may increase the sensitivity of the SPOT assay.

TABLE 6
Clinical evaluation of the SPOT testing system.

| | SPOT | | | | | |
|---|---|---|---|---|---|---|
| | readouts | | | Ct value | | |
| | N | E | qRT-PCR | S | N | ORF |
| ID | gene | gene | interpretation | gene | gene | gene |
| 1 | POS | POS | POS | 27.6 | 26.6 | 27.1 |
| 2 | POS | POS | POS | 26.0 | 24.8 | 25.0 |
| 3 | POS | POS | POS | 21.7 | 18.1 | 20.8 |
| 4 | POS | POS | POS | 19.4 | 17.5 | 19.0 |
| 5 | POS | POS | POS | 27.5 | 26.0 | 27.2 |
| 6 | POS | POS | POS | 26.6 | 22.8 | 25.9 |
| 7 | POS | POS | POS | 29.5 | 27.2 | 29.7 |
| 8 | NEG | NEG | POS | 32.7 | 32.6 | 33.1 |
| 9 | NEG | NEG | NEG | | | |
| 10 | POS | POS | POS | 31.6 | 28.7 | 30.1 |
| 11 | NEG | NEG | NEG | | | |
| 12 | NEG | NEG | NEG | | | |
| 13 | NEG | NEG | NEG | | | |
| 14 | NEG | NEG | NEG | | | |
| 15 | NEG | NEG | NEG | | | |
| 16 | NEG | NEG | NEG | | | |
| 17 | POS | POS | POS | 29.1 | 26.8 | 28.1 |
| 18 | POS | POS | POS | 33.3 | 30.2 | 31.3 |
| 19 | NEG | NEG | NEG | | | |
| 20 | NEG | NEG | NEG | | | |
| 21 | NEG | NEG | NEG | | | |
| 22 | NEG | NEG | POS | 36.5 | 35.6 | 36.6 |
| 23 | POS | POS | POS | 29.5 | 26.6 | 27.8 |
| 24 | NEG | NEG | NEG | | | |
| 25 | NEG | NEG | NEG | | | |
| 26 | NEG | NEG | NEG | | | |
| 27 | POS | POS | POS | 27.9 | 27.6 | 27.6 |
| 28 | NEG | NEG | NEG | | | |
| 29 | NEG | NEG | NEG | | | |
| 31 | NEG | NEG | NEG | | | |
| 32 | POS | POS | POS | 35.1 | 32.0 | neg |
| 33 | POS | POS | POS | 26.5 | 24.9 | 26.0 |
| 34 | POS | POS | POS | 23.7 | 21.9 | 23.4 |
| 36 | POS | POS | POS | 32.1 | 29.9 | 31.6 |
| 37 | POS | POS | POS | 29.3 | 28.1 | 28.8 |
| 38 | POS | POS | POS | 27.3 | 25.9 | 26.5 |
| 39 | POS | POS | POS | 24.6 | 22.9 | 24.8 |
| 40 | NEG | NEG | NEG | | | |
| 41 | NEG | NEG | NEG | | | |
| 42 | POS | POS | POS | 25.6 | 24.5 | 25.9 |
| 43 | POS | POS | POS | 21.4 | 20.8 | 21.4 |
| 44 | NEG | NEG | NEG | | | |
| 45 | POS | POS | POS | 30.5 | 29.3 | 30.8 |
| 46 | NEG | NEG | NEG | | | |
| 47 | POS | POS | POS | 32.3 | 31.3 | 33.6 |
| 48 | NEG | NEG | NEG | | | |
| 49 | NEG | NEG | NEG | | | |
| 50 | POS | POS | POS | 22.2 | 21.2 | 21.9 |
| 51 | NEG | NEG | NEG | | | |
| 52 | NEG | NEG | NEG | | | |
| 53 | NEG | NEG | POS | | | |
| 54 | NEG | NEG | NEG | | | |
| 55 | NEG | NEG | NEG | | | |
| 56 | NEG | NEG | NEG | | | |
| 57 | NEG | NEG | NEG | | | |
| 58 | POS | POS | POS | 25.2 | 23.4 | 25.1 |
| 59 | POS | POS | POS | 22.1 | 21.2 | 21.9 |
| 60 | NEG | NEG | NEG | | | |
| 61 | NEG | NEG | NEG | | | |
| 62 | NEG | NEG | NEG | | | |
| 63 | NEG | NEG | NEG | | | |
| 64 | NEG | NEG | NEG | | | |
| 65 | NEG | NEG | NEG | | | |
| 66 | NEG | NEG | NEG | | | |
| 67 | NEG | NEG | NEG | | | |
| 68 | NEG | NEG | NEG | | | |
| 69 | NEG | NEG | NEG | | | |
| 70 | NEG | NEG | NEG | | | |
| 71 | NEG | NEG | NEG | | | |
| 71 | NEG | NEG | NEG | | | |
| 73 | NEG | NEG | NEG | | | |
| 74 | NEG | NEG | NEG | | | |
| 75 | NEG | NEG | NEG | | | |
| 76 | NEG | POS | NEG | | | |
| 77 | NEG | NEG | NEG | | | |
| 78 | NEG | NEG | NEG | | | |
| 79 | NEG | NEG | NEG | | | |
| 80 | NEG | NEG | NEG | | | |
| 81 | NEG | NEG | NEG | | | |
| 82 | POS | POS | POS | 34.5 | 30.1 | 35.2 |
| 83 | NEG | NEG | NEG | | | |
| 84 | NEG | NEG | NEG | | | |
| 85 | NEG | NEG | NEG | | | |
| 86 | NEG | NEG | NEG | | | |
| 87 | NEG | NEG | NEG | | | |
| 88 | NEG | NEG | NEG | | | |
| 89 | NEG | NEG | NEG | | | |
| 90 | NEG | NEG | NEG | | | |
| 91 | NEG | NEG | NEG | | | |
| 92 | NEG | NEG | NEG | | | |
| 93 | NEG | NEG | NEG | | | |
| 94 | NEG | NEG | NEG | | | |
| 95 | NEG | NEG | NEG | | | |
| 96 | NEG | NEG | NEG | | | |
| 97 | NEG | NEG | NEG | | | |
| 98 | NEG | NEG | NEG | | | |
| 99 | NEG | NEG | NEG | | | |
| 100 | NEG | NEG | NEG | | | |
| 101 | NEG | NEG | NEG | | | |
| 102 | NEG | NEG | NEG | | | |
| 103 | NEG | NEG | NEG | | | |
| 104 | NEG | NEG | NEG | | | |
| 105 | POS | POS | POS | 29.9 | 27.5 | 29.7 |
| 106 | NEG | NEG | NEG | | | |

The SPOT system provides high specificity and accuracy resulting from Ago proteins' unique multiplexing capability. LAMP reactions are prone to non-specific amplification, significantly increasing the rate of false positives and precluding their use as a diagnostic assay. Coupling LAMP with Cas12-based nucleic acid detection reduces these false positives by requiring a single sequence-specific cleavage to generate an output signal. The Ago-based detection method further improves upon specificity by requiring multiple sequence-specific cleavages to generate a positive test result: two cuts with primary guides to release the 16 nt secondary guide, followed by secondary cleavage of the quenched probe. Moreover, Cas12-based methods rely upon collateral activity to indiscriminately cleave quenched probes after recognition of a target site and cannot be used to link multiple specific recognition sites to distinct output signals in a single reaction. In contrast, the sequence-specific cleavage of a quenched probe by PfAgo in the SPOT assay enables multiplexing by linking a unique secondary gDNA to the release of a fluorophore. Multiplexing can not only increase the specificity of detection, but also enable testing for multiple viruses and bacteria or distinguishing between SARS-CoV-2 variants in a single reaction.

The SPOT system also provides the development of a portable, hand-held and battery-powered testing device. The SPOT device is assembled from simple, readily available components: electronics, optical filters, and a heat block. Testing results from the SPOT device can be transferred to a computer or smart phone with a USB cable, enabling centralized data management to support test-trace-isolate protocols. Considering that high testing frequency coupled with quick turnaround time may be more important than test sensitivity to efficiently combat spread of the SARS-CoV-2 virus, portable and low-cost testing systems such as the SPOT system will play a critical role in deploying large-scale rapid and portable diagnostics for this and future pandemics.

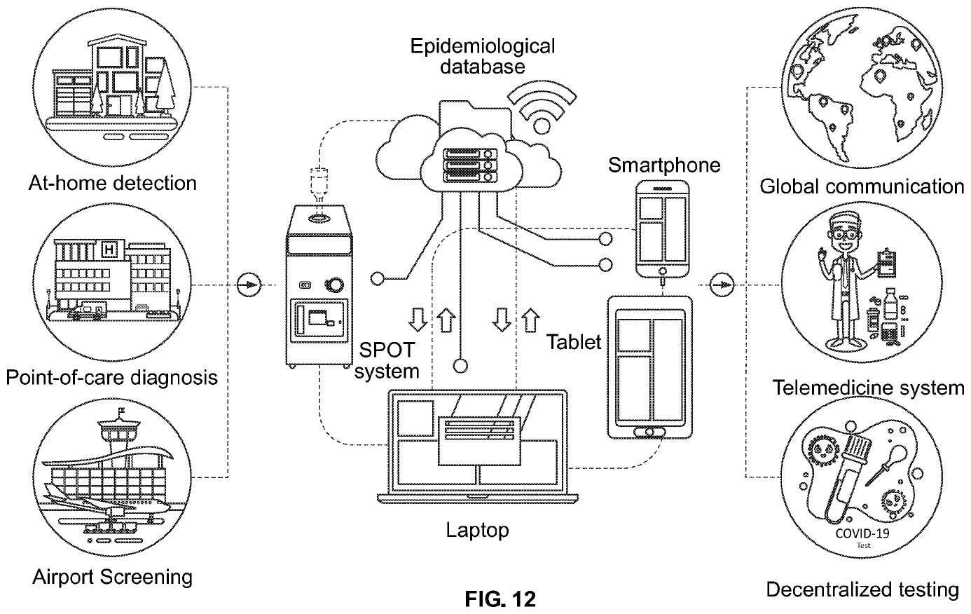

The SPOT system presented here enables rapid diagnosis without trained personnel and only minimal liquid handling. An enlarged heating system and separated module for fluorescent quantification can be used to build a low-cost high throughput SPOT device (1000 tests per day per testing system). The SPOT system can enable rapid and low-cost test-trace-isolate measures with minimal financial investment or training requirements, expanding the availability of testing and serving as a key tool to fight the COVID-19 pandemic. By connecting the SPOT system with internet carriers, the advanced real-time data upload system could endow the SPOT system with functional upgrades to provide better global pandemic controlling, platform of telemedicine, and decentralized testing (FIG. 12). Moreover, this versatile tool can be developed for testing single nucleotide polymorphisms (SNPs) and other pathogens as well.

### REFERENCES

1. Centers for Disease Control and Prevention. Real-time RT-PCR Panel for Detection 2019-nCoV (US Centers for Disease Control and Prevention, 2020); https://www.cdc.gov/coronavirus/2019-ncov/lab/rt-per-detection-instructions.html.
2. Shen, M. et al. Recent advances and perspectives of nucleic acid detection for coronavirus. J. Pharm. Anal. 10, 97-101 (2020)
3. Lalli, M. A. et al. Rapid and extraction-free detection of SARS-CoV-2 from saliva by colorimetric reverse-transcription loop-mediated isothermal amplification. Clin. Chem. (2020). doi.org/10.1093/clinchem/hvaa267
4. Zhang, Y. et al. Rapid molecular detection of SARS-CoV-2 (COVID-19) virus RNA using colorimetric LAMP. medRxiv (2020). doi: httbs://doi.ord/10.1101/2020.02.26.20028373.
5. Broughton, J. P. et al. CRISPR-Cas12-based detection of SARS-CoV-2. Nat. Biotechnol. 38, 870-874 (2020).
6. Joung, J. et al. Point-of-care testing for COVID-19 using SHERLOCK diagnostics. medRxiv (2020). doi: https://doi.org/10.1101/2020.05.04.20091231.
7. Balboni, A. et al. A real-time PCR assay for bat SARS-like coronavirus detection and its application to Italian greater horseshoe bat faecal sample surveys. Sci. World J. 989514 (2012).
8. Enosawa, M. et al. Use of loop-mediated isothermal amplification of the IS900 sequence for rapid detection of cultured Mycobacterium avium subsp. paratuberculosis. J. Clin. Microbil. 41, 4359-4365 (2003).
9. Notomi, T. et al. Loop-mediated isothermal amplification of DNA. Nucleic Acids Res. 28, e63 (2000).

Swarts, D. C. et al. Argonaute of the archaeon Pyrococcus furiosus is a DNA-guided nuclease that targets cognate DNA. Nucleic Acids Res. 43, 5120-5129 (2015).
11. Enghiad, B. & Zhao, H. Programmable DNA-guided artificial restriction enzymes. ACS Synth. Biol. 6, 752-757 (2017).
12. He, R. et al. Pyrococcus furiosus argonaute-mediated nucleic acid detection. Chem. Commun. (Camb) 55, 13219-13222 (2019).
13. Xun, G. et al. The stepwise endonuclease activity of a thermophilic Argonaute protein. bioRxiv, 821280 (2019).
14. To, K. K.-W. et al. Temporal profiles of viral load in posterior oropharyngeal saliva samples and serum antibody responses during infection by SARS-CoV-2: an observational cohort study. Lancet Infect. Dis. 20, 565-574 (2020).
15. To, K. K.-W. et al. Consistent detection of 2019 novel coronavirus in saliva. Clin. Infect. Dis. 71, 841-843 (2020).
16. Ranoa, D. et al. Saliva-Based Molecular Testing for SARS-CoV-2 that Bypasses RNA Extraction. bioRxiv (2020). doi: https://doi.org/10.1101/2020.06.18.159434
17. Kojima, N. et al. Self-collected oral fluid and nasal swab specimens demonstrate comparable sensitivity to clinician-collected nasopharyngeal swab specimens for the detection of SARS-CoV-2. Clin. Infect. Dis. (2020). DOI: https://doi.org/10.1093/cid/ciaa1589
18. Wyllie, A. L. et al. Saliva is more sensitive for SARS-CoV-2 detection in COVID-19 patients than nasopharyngeal swabs. medrxiv (2020). DOI: https://doi.org/10.1101/2020.04.16.20067835
19. Vogels, C. B. et al. SalivaDirect: Simple and sensitive molecular diagnostic test for SARS-CoV-2 surveillance. medRxiv (2020). doi: https://doi.org/10.1101/2020.08.03.20167791
20. Vogels, C. B. F. et al. Analytical sensitivity and efficiency comparisons of SARS-CoV-2 RT-qPCR primer-probe sets. Nat Microbiol 5, 1299-1305 (2020).
21. Larremore, D. B. et al. Test sensitivity is secondary to frequency and turnaround time for COVID-19 screening. Sci. Adv. (2020). DOI:

### SEQUENCES

Oligonucleotide Primers

| Name | | |
|---|---|---|
| Oligonucleotide | | SEQ ID |
| Name | Sequence (5′ → 3′) | NO: |
| F3 2019-nCoV | AACACAAGCTTTCGGCAG | SEQ ID |
| N-gene | | NO: 1 |
| B3 2019-nCoV | GAAATTTGGATCTTTG | SEQ ID |
| N-gene | TCATCC | NO: 2 |
| BIP 2019-nCoV | TGCGGCCAATGTTTGTAAT | SEQ ID |
| N-gene | CAGCCAAGGAAATTTTGGG | NO: 3 |
| | GAC | |
| FIP 2019-nCoV | CGCATTGGCATGGAAGTCA | SEQ ID |
| N-gene | CTTTGATGGCACCTGTGT | NO: 4 |
| | AG | |
| LF 2019-nCoV | TTCCTTGTCTGATTAGTTC | SEQ ID |
| N-gene | | NO: 5 |
| LB 2019-nCoV | ACCTTCGGGAACGTGGTT | SEQ ID |
| N-gene | | NO: 6 |
| F3 2019-nCoV | CCGACGACGACTACTAGC | SEQ ID |
| E-gene | | NO: 7 |
| B3 2019-nCoV | AGAGTAAACGTAAAAAGAA | SEQ ID |
| E-gene | GGTT | NO: 8 |
| BIP 2019-nCoV | ACCTGTCTCTTCCGAAACG | SEQ ID |
| E-gene | AATTTGTAAGCACAAGCTG | NO: 9 |
| | ATG | |
| FIP 2019-nCoV | CTAGCCATCCTTACTGCGC | SEQ ID |
| E-gene | TACTCACGTTAACAATATT | NO: 10 |
| | GCA | |
| LF 2019-nCoV | TCGATTGTGTGCGTACTGC | SEQ ID |
| E-gene | | NO: 11 |
| LB 2019-nCoV | TGAGTACATAAGTTCGTAC | SEQ ID |
| E-gene | | NO: 12 |
| N-gene-FWD IVT | AATTCTAATACGACTCACT | SEQ ID |
| | ATAGGG | NO: 21 |
| N-gene-REV IVT | CACAGTTTGCTGTTTCTTC | SEQ ID |
| | TG | NO: 22 |
| E-gene-FWD IVT | AATTCTAATACGACTCACT | SEQ ID |
| | ATAGGG | NO: 23 |
| E-gene-REV IVT | CTGCCATGGCTAAAATTAAA | SEQ ID |
| | GT | NO: 24 |

Guide DNAs (gDNAs)

| | Sequence | |
|---|---|---|
| Name | (5' → 3') | SEQ ID NO: |
| SARS-COV-2 N | ATGCGCGA | SEQ ID NO: 13 |
| gDNA-1 | CATTCCGA | |
| SARS-COV-2 N | AGAACGCT | SEQ ID NO: 14 |
| gDNA-2 | GAAGCGCT | |
| SARS-COV-2 N | CCCCAGCG | SEQ ID NO: 15 |
| gDNA-3 | CTTCAGCG | |
| SARS-COV-2 E | TGGCTAGT | SEQ ID NO: 16 |
| gDNA-1 | GTAACTAG | |
| SARS-COV-2 E | TGGCTAGT | SEQ ID NO: 17 |
| gDNA-2 | GTAACTAG | |
| SARS-COV-2 E | TTGCTTTC | SEQ ID NO: 18 |
| gDNA-3 | GTGGTATT/ | |

Reporter Molecules

| Name | Sequence (5' → 3') | SEQ ID NO: |
|---|---|---|
| SARS-COV-2 N | TTCCGAAGAACGCTGAA | SEQ ID |
| reporter probe | | NO: 19 |
| SARS-COV-2 E | AACTAGCAAGAATACCA | SEQ ID |
| reporter probe | | NO: 20 |

dsDNA Gene Fragments

| | | SEQ ID |
|---|---|---|
| Name | Sequence (5′ → 3′) | NO: |
| SARS-CoV-2 | AATTCTAATACGACTCACTA | SEQ ID |
| N-gene | TAGGGGTCTGGTAAAGGCCA | NO: 25 |
| | ACAACAACAAGGCCAAACTG | |
| | TCACTAAGAAATCTGCTGCT | |
| | GAGGCTTCTAAGAAGCCTCG | |
| | GCAAAAACGTACTGCCACTA | |
| | AAGCATACAATGTAACACAA | |
| | GCTTTCGGCAGACGTGGTCC | |
| | AGAACAAACCCAAGGAAATT | |
| | TTGGGGACCAGGAACTAATC | |
| | AGACAAGGAACTGATTACAA | |
| | ACATTGGCCGCAAATTGCAC | |
| | AATTTGCCCCCAGCGCTTCA | |
| | GCGTTCTTCGGAATGTCGCG | |
| | CATTGGCATGGAAGTCACAC | |
| | CTTCGGGAACGTGGTTGACC | |
| | TACACAGGTGCCATCAAATT | |
| | GGATGACAAAGATCCAAATT | |
| | TCAAAGATCAAGTCATTTTG | |
| | CTGAATAAGCATATTGACGC | |
| | ATACAAAACATTCCCACCAA | |
| | CAGAGCCTAAAAAGGACAAA | |
| | AAGAAGAAGGCTGATGAAAC | |
| | TCAAGCCTTACCGCAGAGAC | |
| | AGAAGAAACAGCAAACTGTG | |
| SARS-CoV-2 | AATTCTAATACGACTCACTA | SEQ ID |
| E-gene | TAGGGAGACACTGGTGTTGA | NO: 26 |
| | ACATGTTACCTTCTTCATCT | |
| | ACAATAAAATTGTTGATGAG | |
| | CCTGAAGAACATGTCCAAAT | |
| | TCACACAATCGACGGTTCAT | |
| | CCGGAGTTGTTAATCCAGTA | |
| | ATGGAACCAATTTATGATGA | |
| | ACCGACGACGACTACTAGCG | |
| | TGCCTTTGTAAGCACAAGCT | |
| | GATGAGTACGAACTTATGTA | |
| | CTCATTCGTTTCGGAAGAGA | |
| | CAGGTACGTTAATAGTTAAT | |
| | AGCGTACTTCTTTTTCTTGC | |
| | TTTCGTGGTATTCTTGCTAG | |
| | TTACACTAGCCATCCTTACT | |
| | GCGCTTCGATTGTGTGCGTA | |
| | CTGCTGCAATATTGTTAACG | |
| | TGAGTCTTGTAAAACCTTCT | |
| | TTTTACGTTTACTCTCGTGT | |
| | TAAAAATCTGAATTCTTCTA | |
| | GAGTTCCTGATCTTCTGGTC | |
| | TAAACGAACTAAATATTATA | |
| | TTAGTTTTTCTGTTTGGAAC | |
| | TTTAATTTTAGCCATGGCAG | |

What is claimed is:

1. An assay container comprising:
a removable barrier separating an upper chamber from a lower chamber,
the upper chamber comprising one or more sets of primers for the amplification of one or more target nucleic acid molecules and a reverse transcriptase for a reverse transcriptase-loop-mediated isothermal amplification (RT-LAMP) reaction, and
the lower chamber comprising a hyperthermophilic archaeon Argonaute protein, one or more sets of single-stranded guide DNA (gDNA) specific for one or more target nucleic acid molecules, and one or more reporter molecules for a hyperthermophilic archaeon Argonaute protein-based target sequence detection reaction.

2. The assay container of claim 1, wherein the one or more sets of primers comprise two or more oligonucleotides.

3. The assay container of claim 2, wherein the two or more oligonucleotides comprise two or more of SEQ ID NOs:1-6 or SEQ ID NOs:7-12.

4. The assay container of claim 1, wherein the hyperthermophilic archaeon Argonaute protein is an Argonaute protein from Pyrococcus furiosus, Thermococcus thioreducens, Thermococcus onnurineus, Thermococcus eurythermalis, Methanocaldococcus bathoardescens, Methanocaldococcus sp. FS406-22, Methanocaldococcus fervens, Methanocaldococcus jannaschii, Methanotorris formiscicus, Ferroglobus placidus, Archaeoglobus fulgidus, Sulfolobus sp., Methanopyrus kandleri, or Thermogladius cellulolyticus.

5. The assay container of claim 4, wherein the hyperthermophilic archaeon Argonaute protein is a Pyrococcus furiosus Argonaute protein (PfAgo).

6. The assay container of claim 1, wherein the one or more sets of gDNA comprise two or more gDNAs.

7. The assay container of claim 6, wherein the two or more gDNAs comprise two or more of SEQ ID NOs:13-15 or SEQ ID NOs:16-18.

8. The assay container of claim 1, wherein the reporter molecule comprises a nucleic acid molecule having complementarity to the one or more target nucleic acid molecules and a fluorescent moiety.

9. The assay container of claim 8, wherein nucleic acid molecule having complementarity to the target nucleic acid molecule comprises SEQ ID NO:19 or SEQ ID NO:20.

10. The assay container of claim 8, wherein the fluorescent moiety is carboxyfluorescein (FAM) or carboxyrhodamine (ROX).

11. The assay container of claim 1, wherein the one or more target nucleic acid molecules are one or more SARS-CoV-2 nucleic acid molecules.

12. The assay container of claim 11, wherein the SARS-CoV-2 nucleic acid molecule is an envelope (E) nucleic acid molecule, a nucleoprotein (N) nucleic acid molecule, or a combination of both.

13. The assay container of claim 1, wherein the container is a capillary tube.

14. The assay container of claim 1, wherein the removable barrier is a meltable plug.

15. A set of containers comprising: a sample preparation container, and the assay container of claim 1.

16. The set of containers of claim 15, wherein the sample preparation container comprises a DNA extraction solution.

17. The set of containers of claim 15, further comprising a transfer container.

18. A portable diagnostic device comprising:
a) a heating module,
b) a fluorometry module,
c) the assay container of claim 1 within an opening to hold the assay container, and
d) an LCD screen.

19. The device of claim 18, wherein the heating module comprises a copper heat block wrapped with nichrome wire and an integrated fan.

20. The device of claim 19, wherein the copper heat block is configured to allow temperature control, and wherein the integrated fan is configured to enable cooling.

21. The device of claim 18, wherein the fluorometry module comprises: an LED board, an excitation bandpass filter, a photodiode board, emission bandpass filters, and a motherboard comprising a microcontroller.

22. The device of claim 21, wherein the emission bandpass filters are mounted perpendicular to the capillary and filter light to the photodiodes.

23. The device of claim 21, wherein the LED board comprises a blue LED located beneath the excitation bandpass filter, wherein the blue LED shines light into a hole at the bottom of the copper heat block and into the assay container.

24. The device of claim 21, further comprising a transimpedance amplifier configured to convert photodiode current into voltage, which can be read by the microcontroller.

25. The device of claim 21, wherein the photodiode board comprises one or more photodiodes secured behind the emission bandpass filters.

26. The device of claim 18, further comprising a rechargeable and removable lithium-ion battery.

27. The device of claim 18, further comprising a cap covering the device to block excess light.

28. A method of detecting one or more target nucleic acid molecules in a sample comprising:
a) amplifying one or more target nucleic acid molecules using a reverse transcriptase-loop-mediated isothermal amplification (RT-LAMP) reaction to produce amplified target nucleic acid molecules,
b) contacting the amplified target nucleic acid molecules with a hyperthermophilic archaeon Argonaute protein, one or more sets of single-stranded guide DNA (gDNA) specific for the one or more target nucleic acid molecules, and one or more reporter molecules, and
c) detecting fluorescence in the sample,
thereby detecting the target nucleic acid molecules.

29. The method of claim 28, wherein a), b) and c) are performed in a single assay container.

30. The method of claim 28, wherein a), b) and c) are performed in the assay container of claim 1.

31. The method of claim 28, wherein target nucleic acid molecules are one or more SARS-CoV-2 nucleic acid molecules.

32. The method of claim 31, wherein the one or more SARS-CoV-2 nucleic acid molecules are envelope (E) nucleic acid molecules, nucleoprotein (N) nucleic acid molecules, or a combination of both.

33. The method of claim 28, wherein step a) comprises:
a) contacting the sample with the one or more sets of primers specific for amplification of the target nucleic acid molecules and a reverse transcriptase, and
b) heating the sample at 60° C. to 65° C. for 5 or more minutes.

34. The method of claim 33, wherein the one or more sets of primers comprise six oligonucleotides selected from SEQ ID NOs:1-6 and SEQ ID NOs:7-12.

35. The method of claim 28, wherein step b) comprises heating the sample at to 100° C.

36. The method of claim 28, wherein the one or more sets of gDNA comprise three gDNAs selected from SEQ ID NOs:13-15 and SEQ ID NOs:16-18.

37. The method of claim 28, wherein the one or more reporter molecules comprise a nucleic acid molecule having complementarity to the target nucleic acid molecules and a fluorescent moiety.

38. The method of claim 37, wherein the nucleic acid molecules having complementarity to the target nucleic acid molecule comprises SEQ ID NO:19 or SEQ ID NO:20.

39. The method of claim 38, wherein the fluorescent moiety is carboxyfluorescein (FAM) or carboxyrhodamine (ROX).

40. A method of diagnosing a COVID-19 infection in a subject comprising:
detecting one or more SARS-CoV-2 nucleic acid molecules in a sample from the subject comprising:
a) loading the sample into an upper chamber of a assay container comprising a removable barrier separating an upper chamber from a lower chamber, the upper chamber comprising one or more set of primers specific for the amplification of one or more SARS-CoV-2 nucleic acid molecules and the reverse transcriptase for a reverse transcriptase-loop-mediated isothermal amplification (RT-LAMP) reaction and the lower chamber comprising a hyperthermophilic archaeon Argonaute protein, one or more sets of single-stranded guide DNA (gDNA) specific for one or more SARS-CoV-2 nucleic acid molecules, and one or more reporter molecules for a hyperthermophilic archaeon Argonaute protein-based target sequence detection reaction;
b) inserting the assay container into a portable diagnostic device comprising: a heating module, a fluorometry module, an opening to insert and hold the assay container, and an LCD screen;
c) heating the sample at 60° C. to 65° C. for 5 minutes or more;
d) heating the sample at 90° C. to 100° C. for 3 minutes or more; and
d) reading a diagnostic test result on the LCD screen,
thereby diagnosing a COVID-19 infection in the subject.

41. The method of claim 40, wherein the one or more sets of primers comprise six oligonucleotides selected from SEQ ID NOs:1-6 and SEQ ID NOs:7-12.

42. The method of claim 40, wherein the one or more sets of gDNA comprise three gDNAs selected from SEQ ID NOs:13-15 and SEQ ID NOs:16-18.

43. The method of claim 40, wherein the reporter molecule comprises a nucleic acid molecule having complementarity to the SARS-CoV-2 nucleic acid molecules and a fluorescent moiety.

44. The method of claim 43, wherein the nucleic acid molecule having complementarity to the SARS-CoV-2 nucleic acid molecule comprises SEQ ID NO:19 or SEQ ID NO:20.

45. The method of claim 43, wherein the fluorescent moiety is carboxyfluorescein (FAM) or carboxyrhodamine (ROX).

46. The method of claim 40, further comprising contacting the sample with a DNA extraction solution in a sample preparation container and heating the sample in the sample preparation container prior to a).

47. The method of claim 40, wherein the sample is a saliva sample.
